(19) United States
(12) Patent Application Publication
(10) Pub. No.: US 2022/0062439 A1
(43) Pub. Date: Mar. 3, 2022

(54) LOCALIZED ADMINISTRATION OF RNA MOLECULES FOR THERAPY

(71) Applicants: BioNTech RNA Pharmaceuticals GmbH, Mainz (DE); TRON-Translationale Onkologie an der Universitatsmedizin der Johannes Gutenberg-Universitat Mainz ge, Mainz (DE)

(72) Inventor: Ugur Sahin, Mainz (DE)

(21) Appl. No.: 17/421,680

(22) PCT Filed: Jan. 9, 2020
(86) PCT No.: PCT/EP20/50463

§ 371 Date: Jul. 8, 2021

(30) Foreign Application Priority Data
Jan. 10, 2019 (EP) PCT/EP19/50557

(51) Int. Cl. A61K 48/00; A61K 31/417; A61K 38/18; A61P 35/00

(52) U.S. Cl. CPC A61K 48/0075; A61K 31/417; A61K 48/00; A61P 35/00; A61K 38/1866

(57) ABSTRACT

The present invention relates to local delivery to an organ and/or tissue of an agent such as a peptide and/or polypeptide, by administration of an RNA molecule encoding such agent to an afferent blood vessel of the organ and/or tissue. The agent is thus able to provide its biological function, e.g., therapeutic effect, locally and avoid unwanted systemic effects, including any toxicity observed when the agent encoded by the RNA and/or the encoding RNA itself is administered systemically.

### INTRODUCTION

The present invention relates to local delivery to an organ and/or tissue of an agent, such as a peptide and/or polypeptide, by administration of an RNA molecule encoding such agent to an afferent blood vessel of the organ and/or tissue. The agent is thus able to provide its biological function, e.g., therapeutic effect, locally while avoiding unwanted systemic effects, including any toxicity observed when the agent encoded by the RNA and/or the encoding RNA itself is administered systemically.

### BACKGROUND OF THE INVENTION

The introduction of nucleic acids encoding one or more polypeptides or other expressible agents for treating or preventing diseases has been the object of intensive study for quite some time. Such approaches commonly involve the delivery of a nucleic acid molecule to a target cell or organism but vary in the type of nucleic acid, the encoded product to be expressed and the mode of administration. However, attention to safety concerns associated with the administration of such nucleic acids has been increasing in recent years, for example, in view of adverse reactions.

Various approaches have been proposed, including administration of single stranded or double-stranded RNA, in the form of naked RNA, or in complexed or packaged form, e.g., in non-viral or viral delivery vehicles. In viruses and in viral delivery vehicles, the nucleic acid is typically encapsulated by proteins and/or lipids (virus particle). For example, engineered RNA virus particles derived from RNA viruses have been proposed as delivery vehicles for treating plants (WO 2000/053780 A2) or for vaccination of mammals (Tubulekas et al., 1997, Gene 190:191-195). In view of safety concerns, the medical and veterinary community is reluctant to administer RNA virus particles to humans or animals. Non-viral delivery vehicles that could be applicable to RNA have been extensively investigated for development of gene delivery based therapeutics. However, for various reasons translation of non-viral gene delivery approaches into clinical practice has not been very successful, such as reasons associated with unsatisfying levels of gene expression in the target organ, technological and regulatory problems related to pharmaceutical development of such complex products, and safety reasons due to systemic or off-target toxicity.

Thus, there is a need for methods and pharmaceutical products that avoid the adverse reactions to the nucleic acid vehicle and/or the encoded and expressed gene product. As described herein, the present invention addresses this need.

### SUMMARY OF THE INVENTION

The present invention is directed to a method for locally expressing an agent, e.g., a peptide or polypeptide, in an organ or tissue in a subject, comprising administering to an afferent blood vessel of the organ or tissue an RNA, which RNA encodes the agent. In an embodiment, the afferent blood vessel feeds blood directly into the organ or tissue without feeding blood into other organs or tissues. In an embodiment, the afferent blood vessel is proximal/immediately upstream/close to the vascular bed of the organ or tissue. In an embodiment, the afferent blood vessel is a part of the vascular bed of the organ or tissue. In a preferred embodiment, the agent is a peptide or a polypeptide. In certain embodiment, the RNA encodes a single agent or encodes more than one agent. Further, where the RNA encodes more than one agent, the agents can be or the same type, e.g., peptide or polypeptides. In an embodiment, two or more distinct RNA molecules can be administered, in which each distinct RNA molecule encodes a different agent.

In a preferred embodiment, the encoded agent is one that is not suitable for systemic routes of injection, such as intravenous, intradermal, or intraperitoneal administration. For example, the agent is one that is rapidly degraded in the blood stream or shows unacceptable toxicity, e.g., when administered systemically to the subject. In an embodiment, the agent can provide a therapeutic effect or can serve as a detectable moiety. In an exemplary embodiment, the agent is a bacterial toxin, such as tetanus or botulism toxin, which preferably is expressed from the RNA linked to a targeting moiety to target the toxin to a particular cell present in the organ or tissue. In another embodiment the agent is a cytokine or ligand (e.g., TNF-α), which brings the risk of an anaphylactic shock if administered systemically.

In an embodiment, the agent is one that has been modified to have a shorter half-life in the blood stream, for example, the agent is a peptide or polypeptide that has been modified to contain one or more additional protease cleavage sites within its amino acid sequence. In an embodiment, the agent is one that has been modified to be less permeable to a cell membrane, such that when expressed in a cell, the agent is not or is less able to pass through the cell membrane, (e.g., passively secreted out of the cell) compared to the unmodified agent and/or when found in the blood stream, e.g., due to lysis of a cell in which it was expressed, does not enter cells or enters cells with less frequency compared to the unmodified agent.

In an embodiment, the RNA can be administered in combination with a vasoactive agent, for example, a vasoactive agent that enhances transcapillary vesicular transport across the capillary endothelial cell wall. In an exemplary embodiment, the vasoactive agent is histamine or a vascular endothelial growth factor. In an embodiment, the vasoactive agent can be administered before, after or together with the RNA, preferably within about 1, 2, 3, 4, 5, 6, 7, 8, 9, 10, 11, 12, 13, 14, 15 minutes before or after administration of the RNA. In a preferred embodiment, the vasoactive agent is administered to the same afferent blood vessel as the RNA.

In one embodiment, the RNA can be administered in a composition comprising the RNA and a pharmaceutically acceptable carrier.

In an embodiment, the RNA can be comprised in a complex or vesicle. The vesicle can be a multilamellar vesicle, a unilammelar vesicle, or a mixture thereof, e.g., the vesicle can be a liposome. In an embodiment, the complex can be a polyplex particle. Optionally, the complex or vesicle further comprises a ligand for site-specific binding. In an embodiment, the RNA can be naked, i.e., not complexed with protein or comprised within a complex or vesicle or in a viral particle.

In an embodiment, the method can be used in therapy or diagnosis, for example, when the agent is one that provides for a therapeutic effect or when the agent is one that can serve as a detectable moiety.

In an embodiment, the encoded agent can be detected in the organ or tissue but is not substantially detected in other organs or tissues or in the blood stream following administration of the RNA within a certain time period, e.g., 1, 2, 3, 4, 5, 6, 7, 8, 9, 10 or more hours after administration of the RNA. In an embodiment, substantially detected means less than 50%, 45%, 40%, 35%, 30%, 25%, 20%, 15%, 10%, 5%, 4%, 3%, 2%, 1% of the encoded agent can be detected either physically, i.e., immunologically, or by its activity in other organs or tissues or in the blood stream compared to that detected in the organ or tissue in which it was administered via an afferent blood vessel of the organ or tissue. In certain embodiments, the tissue can be tumor tissue. In certain embodiments, the organ can be liver, thyroid, pancreas, kidney, lung, bladder, colon, ovary, testicle, prostate, breast, uterus, heart, stomach, or brain.

The present invention also is directed to a method for treating, preventing or diagnosing a disease in an organ or tissue in a subject, comprising administering to an afferent blood vessel of the organ or tissue an RNA, which RNA encodes an agent that is effective in treating, preventing or diagnosing the disease, e.g., cancer or a tumor. In an embodiment, the tumor is a primary tumor or is a metastasis of a primary tumor.

In an embodiment, the RNA is taken up by the cells of the organ or tissue and the agent is expressed in the cells, e.g., endothelial cells of the organ or tissue, optionally wherein the agent is secreted by the cells. Preferably, the agent is distributed substantially only in the organ or tissue. In an embodiment, the agent is one that is cytotoxic, preferably cytotoxic to cancer cells. In an embodiment, distributed substantially means less than 50%, 45%, 40%, 35%, 30%, 25%, 20%, 15%, 10%, 5%, 4%, 3%, 2%, 1% of the encoded agent can be detected either physically, i.e., immunologically, or by its activity in other organs or tissues or in the blood stream compared to that detected in the organ or tissue in which it was administered via an afferent blood vessel of the organ or tissue.

The present invention also is directed to a pharmaceutical composition comprising RNA encoding an agent which is formulated for administration into an afferent blood vessel, e.g., formulated with a vasoactive agent in a neutral buffer.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the tefins used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (e.g., Green and Sambrook, Molecular Cloning: A Laboratory Manual, 4^{th} Edition, Cold Spring Harbor Laboratory Press, Cold Spring Harbor 2012).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present invention envisions the local administration of an agent, e.g., peptide or polypeptide, to an organ or tissue by administration of an RNA molecule encoding the agent to an afferent blood vessel of the organ or tissue. The afferent blood vessel, e.g., an artery, is one that feeds blood directly into the organ or tissue without feeding blood into other organs or tissues. The afferent blood vessel can be proximal/immediately upstream/close to the vascular bed of the organ or tissue, e.g., upstream of the blood flow to the organ or tissue but not so far upstream that the blood vessel feeds blood into other organs or tissues, e.g., those organs or tissues in which the agent is not intended to be expressed, or such that any toxicity of the encoded agent that would be seen with systemic administration is observed. In an embodiment, the afferent blood vessel can be a part of the vascular bed of the organ or tissue.

Administration to the afferent blood vessel can be by any means known to the skilled practitioner. For example, a needle can be inserted into a site in an afferent blood vessel and the RNA can be injected (released from the delivery device) into the blood stream at that site. In certain embodiments, the afferent blood vessel and/or the site of insertion can be determined using known methods to identify afferent blood vessels, such as contrast imaging (e.g., angiography) or measuring blood flow or oxygenation levels of the blood in a blood vessel suspected of being an afferent blood vessel of a particular organ or tissue.

In an embodiment, administration to an afferent blood vessel does not solely encompass administering directly at the site where a delivery device containing the RNA, such as a cannula, catheter or syringe, enters the blood vessel and releases the RNA at that site, but also encompasses releasing the RNA from the delivery device at a desired site in the afferent blood vessel, which desired site is a different site from where the delivery device entered/pierced the blood vessel. In other words, the delivery device can enter a different blood vessel or the same afferent blood vessel at a different location than the desired site where the RNA is to be released into the blood stream of the afferent blood vessel. For example, the delivery device, such as a catheter or microcatheter, optionally with a guide wire, is placed in an artery and with attention to the angioanatomy and blood flow pattern of the subject, the end of the catheter is moved to the desired site in the afferent blood vessel of the organ or tissue and the RNA is injected/released into the blood stream at that desired site. The positioning of the catheter and/or the verification of its position can be done in connection with the use of a contrast agent (angiogram). For example, where the organ is liver, a catheter can be inserted in the femoral artery in the leg and is then guided to a site in an afferent blood vessel directly feeding blood into the liver and the RNA can be released into the blood stream at that site.

The agent can be any molecule that can be encoded and expressed by an RNA molecule in a cell, preferably a mammalian cell. In a preferred embodiment, the agent is a peptide or polypeptide and, thus, the peptide or polypeptide encoded by the RNA can be any peptide or polypeptide that is able to be translated from the RNA in a cell of the tissue or organ. For example, the encoded peptide or polypeptide can have an activity that disrupts the ability of a cell to grow and divide, or causes the cell to die by lysing the cell. In an embodiment, the encoded peptide or polypeptide can have an activity that enhances cell growth or regeneration. Further, since the translation of the RNA can be independent of cell division/mitosis and transcription, the expressed peptide or polypeptide can also affect cells that are not actively growing, i.e., quiescent or post-mitotic cells, by being translated in the cell cytoplasm, e.g., independent of DNA replication or the cell cycle. Exemplary peptides or polypeptides are those that are known to be useful in therapeutic/prophylactic/diagnostic applications and, thus, useful in the therapeutic/prophylactic/diagnostic methods of the invention, including those peptides/polypeptides too toxic to an organism as a whole to be administered systemically.

In an embodiment, the agent expressed by the RNA can be modified to increase or decrease its permeability, for example, a peptide or polypeptide can be modified to change its vascular and/or cell permeability. As used herein, permeability is the ability to cross a barrier, such as a biological membrane, preferably in a passive manner. The barrier can be a cell membrane or another type of membrane separating cells or tissues, such as endothelial tissues/organs, from each other. Such membranes include the membrane surrounding an organ, i.e., the visceral or serous membrane, for example, the pericardium or epicardium membrane, the pleura, or peritoneal membrane.

One type of permeability is cell permeability, which is the ability to cross the cell membrane, preferably passively. Another type of permeability is vascular permeability, which is often in the form of capillary permeability or microvascular permeability, and is characterized by the capacity of a blood vessel wall to allow for the flow of small molecules (drugs, nutrients, water, ions) or even whole cells (lymphocytes on their way to the site of inflammation) in and out of the blood vessel, also preferably passively. Blood vessel walls are lined by a single layer of endothelial cells and the gaps between endothelial cells (cell junctions) are strictly regulated depending on the type and physiological state of the tissue. There are several techniques known in the art to measure vascular permeability. For example, the perfusion of microvessels with a micropipette and measuring the velocity of cells, see, e.g., Bates et al., 2002, Vascul. Pharmacol. 39:225-237. Another technique uses multiphoton fluorescence intravital microscopy, see, e.g., Reyes-Aldasoro et al., 2008, Microcirculation 15:65-79.

Preferably, the agent is modified to be less permeable. In this manner, the agent can be trapped in the cell (or tissue) in which it is expressed. This can be important for extracellularly effective or normally secreted agents such as cytokines, which, in this embodiment, would be modified to decrease their permeability in order to trap them in the cell in which they are expressed and/or in a target tissue.

In an embodiment, the agent is one that has been modified to be less permeable to a cell membrane, such that when expressed in a cell, the agent is not or is less able to pass through the cell membrane, (e.g., passively secreted out of the cell) compared to the unmodified agent and/or when found in the blood stream, e.g., due to lysis of a cell in which it was expressed, does not enter cells or enters cells with less frequency compared to the unmodified agent. It is known in the art that there are certain characteristics which lead to increased permeability, such as the degree of rigidity of a molecule. Thus, an agent, preferably a peptide or polypeptide, can be modified to be less rigid, e.g., decrease its helicity, in order to decrease its permeability. Other modifications can include removal of intramolecular hydrogen bonds to increase flexibility, and/or removal of N-methylation sites.

For example, a peptide or polypeptide encoded by an RNA molecule described herein can be a bacterial toxin, such as tetanus, botulinum, Pseudomonas exotoxin and diphtheria toxin, see e.g., Johnson, 1999, Annu. Rev. Microbiol. 53:551-575 and Turton et al., 2002, TRENDS Biochem. Sci. 27:552-558 disclosing various bacterial toxins and their use in treating a variety of diseases and disorders. Other toxins include lethal toxin from B. anthracis, Pertussis toxin from B. pertussis and cytotoxic necrotizing factor I from E. coli, see, e.g., Fabbri et al., 2008, Cuff. Medicinal Chem. 15:1116-1125. In an embodiment, the peptide or polypeptide encoded by the RNA molecule is a hybrid cytotoxic protein, such as Pseudomonas exotoxin or diphtheria toxin fused to a sequence that binds to a target cell to be killed, such as Pseudomonas exotoxin fused to TGF-α which binds and kills cells expressing the TGF-α receptor; see, e.g., Pastan and FitzGerald, 1991, Science 254:1173-1177 disclosing a number of hybrid cytotoxic proteins and exemplary diseases that can be treated using such hybrid cytotoxic proteins. Other examples include a recombinant toxin comprising human interleukin-2 (IL-2) and truncated diphtheria toxin (DAB₃₈₉-IL-2, denileukin diftitox, or ONTAK®, Seragen, Inc.) useful in the treatment of lymphoma, and a recombinant toxin comprising a truncated Pseudomonas exotoxin, P38, fused to an antibody fragment that binds CD22, which is useful in the treatment of lymphoma and leukemia, see, e.g., Kreitman, 2003, Curr. Opin. Mol. Therap. 5:44-51.

In one embodiment, the RNA is administered in combination with a vasoactive agent, at the same time or within about 1, 2, 3, 4, 5, 6, 7, 8, 9, 10, 11, 12, 13, 14, 15 minutes before or after administration of the RNA. The vasoactive agent can enhance the entry of the RNA into the cells of the tissue or organ. A vasoactive agent, as used herein, refers to a natural or synthetic substance that induces increased vascular permeability and enhances transfer of macromolecules such as RNA molecules across capillary walls. By augmenting vascular permeability to macromolecules such as RNA or otherwise facilitating the transfer of macromolecules into the capillary bed perfused by an artery, vasoactive agents can enhance delivery of these macromolecules to the targeted sites and thus in connection with the present invention effectively enhance overall expression of the RNA molecule in the target tissue or organ. Histamine is an exemplary vasoactive agent, along with histamine derivatives and agonists such as those that interact with the histamine H receptor, such as 2-methyl-histamine, 2-pyridyl-ethylamine, betahistine, and 2-thiazolyl-ethylamine. These and additional histamine agonists are described, for example, in Goodman and Gilman's The Pharmacological Basis of Therapeutics (12^{th} Ed., Brunton et al., Eds.) McGraw-Hill, 2012, pp 911-936.

In addition to histamine and histamine agonists, vascular endothelial growth factors (VEGFs) and VEGF agonists can also induce increased vascular permeability and can therefore be used as a vasoactive agent to enhance delivery of the RNA molecule in the context of the compositions and methods of the invention described herein. Human VEGFs have been described, for example, by Tischer et al., 1991, J. Biol. Chem. 206:11947-11954, and references therein; and by Muhlhauser et al., 1995, Cir. Res. 77:1077-10786.

Vasoactive agents can be introduced coincident with administration of the RNA molecule, but are preferably introduced to the target site (e.g., by pre-infusion) within several minutes prior to the introduction of the RNA molecule so that the vasoactive agent is able to elicit a response at the injection/release site prior to exposure of the cells to the RNA molecule.

Diseases and disorders that can be treated and/or prevented according to the invention described herein depend on the agent and its therapeutic/prophylactic activity. For example, where the disease is cancer or a hyperproliferative disease, the agent, e.g., peptide or polypeptide, encoded by the RNA will have an activity that kills or inhibits the growth of the cells. In another example, where the disease is a neurological disease, the agent has an activity to enhance nerve growth or activity or inhibit nerve activity, such as a bacterial toxin to treat a focal dystonia, spasticity, tremor, migraine, or tension headache or a nerve growth factor to promote nerve regeneration. In an embodiment, the agent encoded by the RNA molecule can be a protease and is expressed within the cell. Also, the protease can be one that is specific to proteolytically destroying amyloid plagues, e.g., neprilysn, such that the RNA encoding such a protease can be used to treat Alzheimer's disease. Other agents can include catalytic antibodies to bind and degrade amyloidogenic proteins, see, e.g., Eisele et al., 2015, Nat. Rev. Drug Discov. 14:759-780. In an embodiment, where the disease or disorder can be treated by promoting angiogenesis, the agent can be an agent that promotes the growth of blood vessels, i.e., an angiogenic factor, such as a member of the fibroblast growth factor family, e.g., FGF-1, -2, vascular endothelial growth factor (VEGF), Angiopoietin 1, Angiopoietin 2, PDGF, AC133.

Since administration of the RNA encoding the agent is effectively local to the organ or tissue and the cells contained therein, the cell killing activity can be one that is not specific to the cells of the tissue or organ or cancer cells contained within the tissue or organ. In an embodiment, the cell killing activity of the agent is one that is judged by physicians or other skilled persons to be too toxic/harmful to the patient as a whole such that the RNA encoding such an agent should not be administered systemically, e.g., intravenously. This also applies to where the activity of the encoded agent, e.g., binding to a particular receptor expressed on the cell surface or growth promoting activity, is judged to be too toxic/harmful to the patient as a whole for the RNA encoding the agent to be administered systemically. In an exemplary embodiment, where the agent is one that is too toxic to a particular organ or tissue to be administered systemically but is nevertheless useful in treating a disease in another organ or tissue, the RNA encoding the agent is particularly suitable for administration in an afferent blood vessel of the other organ or tissue in accordance with the present invention.

Other encoded agents include antibodies and functional fragments or derivatives thereof, such as chimeric or humanized antibodies. For example, full length antibodies, as well as functional fragments such as Fv, scFv, Fab, F(ab′)2, F(ab′), scFv-Fc type or diabodies, which generally have the same specificity of binding as the antibody from which they are derived, can be encoded by the RNA molecule.

In one embodiment, the antibody encoded by the RNA is an intrabody, i.e., an antibody expressed by the cell but not secreted such that it binds its target intracellularly. Intrabodies in the context of the present invention can include any antibody or antibody fragment but where such is intracellularly expressed. Intrabodies can be localized and expressed at certain sites in the cell. For example, an intrabody can be expressed in the cytoplasm, which allows for the inhibition of cytoplasmic proteins. By additional coding of a C-terminal ER retention signal (for example KDEL) by the RNA molecule encoding the intrabody, the intrabody can remain in the ER, where it may bind to specific protein (antigen) located in the ER and prevent secretion of the antigen and/or transport of the antigen to the plasma membrane.

Humanized antibodies in the context of the present invention are antibodies in which the constant and variable domains of the non-human antibodies, with the exception of the hypervariable regions, have been replaced by human sequences.

In an embodiment, the RNA encodes or can additionally encode a reporter protein. Certain genes may be chosen as reporters because the characteristics they confer on cells or organisms expressing them may be readily identified and measured, or because they are selectable markers. Reporter genes are often used as an indication of whether a certain gene has been taken up by or expressed in the cell or organism population. Preferably, the expression product of the reporter gene is visually detectable. Common visually detectable reporter proteins typically possess fluorescent or luminescent proteins. Examples of specific reporter genes include the gene that encodes jellyfish green fluorescent protein (GFP), which causes cells that express it to glow green under blue light, the enzyme luciferase, which catalyzes a reaction with luciferin to produce light, and the red fluorescent protein (RFP). Variants of any of these specific reporter genes are possible, as long as the variants possess visually detectable properties. For example, eGFP is a point mutant variant of GFP.

In certain embodiments of the invention, the agent encoded by the RNA is a small interfering RNA (siRNA), which siRNA can interfere with mRNA translation by binding to mRNA or by binding to RNA molecules involved in translation, such as tRNA or RNA components of ribosomes. The actual sequence of the siRNA will depend on the RNA molecule it is designed to bind to such that translation of the RNA molecule is inhibited or such that the biological activity of the bound RNA is inhibited. Exemplary siRNA molecules are described in U.S. Pat. Nos. 7,691,997 B2 and 8,101,741 B2; see also, Resnier et al., 2013, Biomaterials 34:6429-6443 for a review on the use of siRNA in the treatment of cancer.

The term “nucleic acid” comprises deoxyribonucleic acid (DNA), ribonucleic acid (RNA), and locked nucleic acid (LNA). Nucleic acids comprise genomic DNA, cDNA, mRNA, viral RNA, recombinantly prepared and chemically synthesized molecules. According to the invention, a nucleic acid may be in the form of a single stranded or double-stranded and linear or covalently closed circular molecule. The term “nucleic acid” according to the invention also comprises a chemical derivatization of a nucleic acid on a nucleotide base, on the sugar or on the phosphate, and nucleic acids containing non-natural nucleotides and nucleotide analogs. The nucleic acids described may be isolated and/or recombinant nucleic acids.

The term “isolated” as used herein, is intended to refer to a molecule which is substantially free of other molecules such as other cellular material. The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “recombinant” in the context of the present invention means “made through genetic engineering”. Preferably, a “recombinant object” in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the invention “nucleic acid sequence” refers to the sequence of nucleotides in a nucleic acid, e.g., a ribonucleic acid (RNA) or a deoxyribonucleic acid (DNA). The term may refer to an entire nucleic acid molecule (such as to the single strand of an entire nucleic acid molecule) or to a part (e.g., a fragment) thereof.

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5” of that second element. For a double-stranded nucleic acid molecule, indications like “upstream” and “downstream” are given with respect to the (+) strand.

According to the invention, the term “gene” refers to a particular nucleic acid sequence which is responsible for producing one or more cellular products and/or for achieving one or more intercellular or intracellular functions. More specifically, said term relates to a nucleic acid section (DNA or RNA) which comprises a nucleic acid coding for a specific protein or a functional or structural RNA molecule.

The term “vector” is used herein its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

In the context of the present invention, the term “RNA” relates to a molecule which comprises ribonucleotide residues and preferably being entirely or substantially composed of ribonucleotide residues and comprises all RNA types described herein. The term “ribonucleotide” relates to a nucleotide with a hydroxyl group at the 2′-position of a β-D-ribofuranosylgroup. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally-occurring RNAs. The RNA used according to the present invention may have a known composition, or the composition of the RNA may be partially or entirely unknown.

According to the invention, “double-stranded RNA” or “dsRNA” means RNA with two partially or completely complementary strands.

According to the invention, the nucleic acid is preferably single stranded RNA (ssRNA). The term “single stranded RNA” generally refers to an RNA molecule to which no complementary nucleic acid molecule (typically no complementary RNA molecule) is associated. Single stranded RNA may contain self-complementary sequences that allow parts of the RNA to fold back and to form secondary structure motifs including without limitation base pairs, stems, stem loops and bulges. Single stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

Particularly preferred single stranded RNA according to the invention is mRNA and replicon-RNA such as self-replicating RNA. According to the present invention, the RNA can be coding RNA, i.e., RNA encoding an agent such as a peptide or polypeptide. Preferably, the RNA is pharmaceutically active RNA.

A “pharmaceutically active RNA” is an RNA that encodes a pharmaceutically active agent, such as a peptide or polypeptide including a cytotoxic polypeptide or is pharmaceutically active in its own, e.g., it has one or more pharmaceutical activities such as those described for pharmaceutically active proteins.

According to the invention, the term “RNA encoding a peptide or polypeptide” means that the RNA, if present in the appropriate environment, preferably within a cell, can direct the assembly of amino acids to produce the peptide or polypeptide, during the process of translation. Preferably, coding RNA according to the invention is able to interact with the cellular translation machinery allowing translation of the coding RNA to yield an agent according to the present invention. Similarly, the term “RNA encoding an agent” means that the RNA, if present in the appropriate environment, preferably within a cell, can direct the assembly of nucleotides, e.g., ribonucleotides, or amino acids to produce the agent, during the process of translation or transcription.

According to the invention, the term “mRNA” means “messenger-RNA” and relates to a transcript which is typically generated by using a DNA template and encodes, e.g., a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to the skilled person. For example, there is a variety of in vitro transcription kits commercially available.

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) tail. Thus, the 3′-UTR is upstream of the poly(A) tail (if present), e.g., directly adjacent to the poly(A) tail. A 5′-UTR, if present, is located at the 5′ end of a gene, upstream of the start codon of a protein-encoding region. A 5′-UTR is downstream of the 5′-cap (if present), e.g., directly adjacent to the 5′-cap. 5′- and/or 3′-untranslated regions may, according to the invention, be functionally linked to an open reading frame, so as for these regions to be associated with the open reading frame in such a way that the stability and/or translation efficiency of the RNA comprising said open reading frame are increased.

According to the invention, the terms “poly(A) sequence” or “poly(A) tail” refer to an uninterrupted or interrupted sequence of adenylate residues which is typically located at the 3′ end of an RNA molecule. An uninterrupted sequence is characterized by consecutive adenylate residues. In nature, an uninterrupted poly(A) sequence is typical. While a poly(A) sequence is normally not encoded in eukaryotic DNA, but is attached during eukaryotic transcription in the cell nucleus to the free 3′ end of the RNA by a template-independent RNA polymerase after transcription, the present invention encompasses poly(A) sequences encoded by DNA.

Terms such as “5′-cap”, “cap”, “5′-cap structure”, or “cap structure” are used synonymously to refer to a dinucleotide that is found on the 5′ end of some eukaryotic primary transcripts such as precursor messenger RNA. A 5′-cap is a structure wherein a (optionally modified) guanosine is bonded to the first nucleotide of an mRNA molecule via a 5′ to 5′ triphosphate linkage (or modified triphosphate linkage in the case of certain cap analogs). The terms can refer to a conventional cap or to a cap analog.

RNA molecules according to the invention may be characterized by a 5′-cap, a 5′-UTR, a 3′-UTR, a poly(A) sequence, and/or adaptation of the codon usage.

RNA molecules for use according to the methods of the invention can have any size (length) that is sufficient to allow for expression of the encoded agent once the RNA has entered a cell. Thus, the size of the RNA molecule will depend on the size of the encoded agent and the necessary regulatory sequences for translation. For example, the size of the RNA molecule can vary from 50 to 20000 bases. In an embodiment, the size is in a range from 100 to 2000 bases, more preferably from 500 to 1500 bases. Other RNA molecules for use according to the methods of the invention preferably have a size of more than 2000 bases, preferably more than 3000 bases, more than 4000 bases, more than 5000 bases, more than 6000 bases, more than 7000 bases, more than 8000 bases, more than 9000 bases, or more than 10000 bases. RNA molecules for use according to the invention preferably have a size of 6000 to 20000 bases, preferably 6000 to 15000 bases, preferably 9000 to 12000 bases.

The term “translation efficiency” relates to the amount of translation product provided by an RNA molecule within a particular period of time.

The term “modification” in the context of the RNA used in the present invention includes any modification of an RNA which is not naturally present in said RNA.

The term “stability” of an agent, e.g., RNA, peptides or polypeptides relates to the “half-life” of the agent, e.g., the RNA, peptides or polypeptides, respectively. “Half-life” relates to the period of time which is needed to eliminate half of the maximum (pharmacologic) activity, amount, or number of molecules of an agent, e.g., from the body or blood of a patient to which the agent was administered or as measured in an in vitro assay. The maximum pharmacologic activity is defined by the steady state value, where intake equals elimination. Since the kinetics of certain agents, such as pharmaceutical drugs, can be complex, the “half-life” of an agent does not necessarily follow or is limited to first order kinetics.

In the context of the present invention, the half-life of an agent, e.g., RNA, peptides or polypeptides, can be indicative of the stability of said RNA, peptides or polypeptides, respectively. For example, the half-life of an agent expressed from an RNA may be influenced by the “duration of expression” of the RNA. It can be expected that RNA having a long half-life will be expressed for an extended time period and one having shorter half-life will be expressed for a shorter time period. Also, the half-life of peptides or polypeptides can be influenced by the duration of therapeutic effect of such peptides or polypeptides. As used herein, half-life in blood and/or serum reflects how quickly the administered material, e.g., the RNA or its encoded product, is degraded, i.e., loses its biological activity, e.g., reflects how quickly it is degraded by enzymes in the blood and/or serum, as well as how quickly it is removed from the blood by the kidneys. Thus, in one embodiment, in order to decrease the half-life of the RNA or its encoded product, renal efficiency can be increased by means known in the art, e.g., co-administration of a diuretic. The half-life also can be reflected by the dose response curve, which is the relationship between the pharmacologic activity and the amount, or number of molecules of an agent, e.g., a drug.

According to the invention, the stability and translation efficiency of RNA may be modified as required. In an embodiment, the RNA is modified to be less stable in the blood and/or serum, i.e., is degraded in a shorter time period than the parental unmodified RNA. In another embodiment, the RNA is modified to be more stable in the blood and/or serum. For example, RNA may be stabilized and its translation increased by one or more modifications having a stabilizing effects and/or increasing translation efficiency of RNA. For example, it is known that in order to increase expression of RNA, it may be modified within the coding region, i.e., the sequence encoding the expressed peptide or protein, preferably without altering the sequence of the expressed peptide or protein, so as to increase the GC-content to increase mRNA stability. Thus, in an embodiment, the GC-content of the mRNA can be increased or decreased in order to make the RNA more or less stable, as desired. In an embodiment, mRNA may be modified by stabilizing modifications and capping or may be modified to make it less stable in the blood.

In one embodiment, the WI n “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap (m⁷G). In the context of the present invention, the term “5′-cap” includes a 5′-cap analog that resembles the RNA cap structure and is modified to enhance translation of RNA if attached thereto, preferably in vivo and/or in a cell.

In one embodiment of the invention, the RNA used according to the invention has uncapped 5′-triphosphates. Removal of such uncapped 5′-triphosphates can be achieved by treating RNA with a phosphatase.

The RNA may comprise further modifications. For example, a further modification of the RNA used in the present invention may be an extension or truncation of the naturally occurring poly(A) tail or an alteration of the 5′- or 3′-untranslated regions (UTR) such as introduction of a UTR which is not related to the coding region of said RNA.

RNA having an unmasked poly-A sequence is translated more efficiently than RNA having a masked poly-A sequence. The term “poly(A) tail” or “poly-A sequence” relates to a sequence of adenyl (A) residues which typically is located on the 3′-end of an RNA molecule and “unmasked poly-A sequence” means that the poly-A sequence at the 3′ end of an RNA molecule ends with an A of the poly-A sequence and is not followed by nucleotides other than A located at the 3′ end, i.e., downstream, of the poly-A sequence. Furthermore, a long poly-A sequence of about 120 base pairs results in an optimal translation efficiency of RNA.

Therefore, in order to increase expression of the RNA used according to the present invention, it may be modified so as to be present in conjunction with a poly-A sequence, preferably having a length of 10 to 500, more preferably 30 to 300, even more preferably 65 to 200 and especially 100 to 150 adenosine residues. In an especially preferred embodiment the poly-A sequence has a length of approximately 120 adenosine residues. To further increase expression of the RNA used according to the invention, the poly-A sequence can be unmasked.

Of course, if according to the present invention it is desired to decrease stability and/or translation efficiency of RNA, it is possible to modify RNA so as to interfere with the function of elements as described above increasing the stability and/or translation efficiency of RNA. For example, the 5′ cap can be removed and/or the poly-A tail can be masked or removed from the RNA.

In an embodiment, the RNA according to the invention may have modified ribonucleotides in order to decrease cytotoxicity. For example, in one embodiment, in the RNA used according to the invention 5-methylcytidine is substituted partially or completely, preferably completely, for cytidine. Alternatively or additionally, in one embodiment, in the RNA used according to the invention pseudouridine is substituted partially or completely, preferably completely, for uridine.

In an embodiment, the RNA to be administered according to the invention is non-immunogenic.

The term “non-immunogenic RNA” as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic. In an embodiment, non-immunogenic RNA is rendered non-immunogenic by incorporating modified nucleotides suppressing RNA-mediated activation of innate immune receptors into the RNA and removing double-stranded RNA (dsRNA).

For rendering the non-immunogenic RNA non-immunogenic by the incorporation of modified nucleotides, any modified nucleotide may be used as long as it lowers or suppresses immunogenicity of the RNA. Particularly preferred are modified nucleotides that suppress RNA-mediated activation of innate immune receptors. In one embodiment, the modified nucleotides comprises a replacement of one or more uridines with a nucleoside comprising a modified nucleobase. In one embodiment, the modified nucleobase is a modified uracil. In one embodiment, the nucleoside comprising a modified nucleobase is selected from the group consisting of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5 s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine (τm5 s2U), 1-taurinomethyl-4-thio-pseudouridine, 5-methyl-2-thio-uridine (m5 s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 ψ), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In one particularly preferred embodiment, the nucleoside comprising a modified nucleobase is pseudouridine (ψ), N1-methyl-pseudouridine (m1ψ) or 5-methyl-uridine (m5U), in particular 1-methyl-pseudouridine.

The structure of an exemplary nucleoside comprising a modified nucleobase is 1-methylpseudouridine m1Ψ:

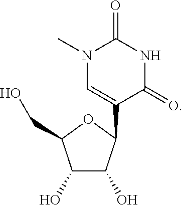 [SMILES: Cn1cc([C@@H]2O[C@H](CO)[C@@H](O)[C@H]2O)c(=O)[nH]c1=O]

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In one embodiment, an RNA preparation is contacted with a cellulose material and the ssRNA is separated from the cellulose material under conditions which allow binding of dsRNA to the cellulose material and do not allow binding of ssRNA to the cellulose material.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

According to the invention, the term “expression” is used in its most general meaning and comprises production of RNA and/or protein. It also comprises partial expression of nucleic acids. Furthermore, expression may be transient or stable. With respect to RNA, the term “expression” or “translation” relates to the process in the ribosomes of a cell by which a strand of coding RNA (e.g., messenger RNA) directs the assembly of a sequence of ribonucleotides to make a siRNA or a sequence of amino acids to make a peptide or protein.

The terms “transcription” and “transcribing” relate to a process during which a nucleic acid molecule with a particular nucleic acid sequence (the “nucleic acid template”) is read by an RNA polymerase so that the RNA polymerase produces a single stranded RNA molecule. During transcription, the genetic information in a nucleic acid template is transcribed. The nucleic acid template may be DNA; however, e.g., in the case of transcription from an alphaviral nucleic acid template, the template is typically RNA. Subsequently, the transcribed RNA may be translated, e.g., into protein. According to the present invention, the term “transcription” comprises “in vitro transcription”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present invention encompassed by the term “vector”. The cloning vectors are preferably plasmids. According to the present invention, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The single stranded nucleic acid molecule produced during transcription typically has a nucleic acid sequence that is the complementary sequence of the template.

According to the invention, the terms “template” or “nucleic acid template” or “template nucleic acid” generally refer to a nucleic acid sequence that may be replicated or transcribed.

The term “expression control sequence” comprises according to the invention promoters, ribosome-binding sequences and other control elements which control transcription of a gene or translation of the derived RNA. In particular embodiments of the invention, the expression control sequences can be regulated. The precise structure of expression control sequences may vary depending on the species or cell type but usually includes 5′-untranscribed and 5′- and 3′-untranslated sequences involved in initiating transcription and translation, respectively. More specifically, 5′-untranscribed expression control sequences include a promoter region which encompasses a promoter sequence for transcription control of the functionally linked gene. Expression control sequences may also include enhancer sequences or upstream activator sequences. An expression control sequence of a DNA molecule usually includes 5′-untranscribed and 5′- and 3′-untranslated sequences such as TATA box, capping sequence, CAAT sequence and the like. An expression control sequence of alphaviral RNA may include a subgenomic promoter and/or one or more conserved sequence element(s). A specific expression control sequence according to the present invention is a subgenomic promoter of an alphavirus, as described herein.

The term “promoter” or “promoter region” refers to a nucleic acid sequence which controls synthesis of a transcript, e.g., a transcript comprising a coding sequence, by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. A specific promoter according to the present invention is a subgenomic promoter of an alphavirus, as described herein. Other specific promoters are genomic plus-strand or negative-strand promoters of an alphavirus.

The term “core promoter” refers to a nucleic acid sequence that is comprised by the promoter. The core promoter is typically the minimal portion of the promoter required to properly initiate transcription. The core promoter typically includes the transcription start site and a binding site for RNA polymerase.

The nucleic acid sequences specified herein, in particular transcribable and coding nucleic acid sequences, may be combined with any expression control sequences which may be homologous or heterologous to said nucleic acid sequences, with the term “homologous” referring to the fact that a nucleic acid sequence is also functionally linked naturally to the expression control sequence, and the term “heterologous” referring to the fact that a nucleic acid sequence is not naturally functionally linked to the expression control sequence.

A nucleic acid sequence, in particular a nucleic acid sequence coding for a peptide or polypeptide, and an expression control sequence are “functionally” linked to one another, if they are covalently linked to one another in such a way that transcription or expression of the transcribable and/or coding nucleic acid sequence is under the control or under the influence of the expression control sequence.

According to the invention, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences.

In particular embodiments, a nucleic acid is functionally linked according to the invention to expression control sequences which may be homologous or heterologous with respect to the nucleic acid.

A “polymerase” generally refers to a molecular entity capable of catalyzing the synthesis of a polymeric molecule from monomeric building blocks. A “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, an RNA polymerase synthesizes an RNA molecule based on a template nucleic acid, which is either a DNA molecule (in that case the RNA polymerase is a DNA-dependent RNA polymerase, DdRP), or is an RNA molecule (in that case the RNA polymerase is an RNA-dependent RNA polymerase, RdRP).

A “RNA-dependent RNA polymerase” or “RdRP”, is an enzyme that catalyzes the transcription of RNA from an RNA template. In the case of alphaviral RNA-dependent RNA polymerase, sequential synthesis of (−) strand complement of genomic RNA and of (+) strand genomic RNA leads to RNA replication. Alphaviral RNA-dependent RNA polymerase is thus synonymously referred to as “RNA replicase”. In nature, RNA-dependent RNA polymerases are typically encoded by all RNA viruses except retroviruses. Typical representatives of viruses encoding an RNA-dependent RNA polymerase are alphaviruses.

According to the present invention, “RNA replication” generally refers to an RNA molecule synthesized based on the nucleotide sequence of a given RNA molecule (template RNA molecule). The RNA molecule that is synthesized may be e.g., identical or complementary to the template RNA molecule. In general, RNA replication may occur via synthesis of a DNA intermediate, or may occur directly by RNA-dependent RNA replication mediated by an RNA-dependent RNA polymerase (RdRP). In the case of alphaviruses, RNA replication does not occur via a DNA intermediate, but is mediated by an RNA-dependent RNA polymerase (RdRP): a template RNA strand (first RNA strand)—or a part thereof—serves as template for the synthesis of a second RNA strand that is complementary to the first RNA strand or to a part thereof. The second RNA strand—or a part thereof—may in turn optionally serve as a template for synthesis of a third RNA strand that is complementary to the second RNA strand or to a part thereof. Thereby, the third RNA strand is identical to the first RNA strand or to a part thereof. Thus, RNA-dependent RNA polymerase is capable of directly synthesizing a complementary RNA strand of a template, and of indirectly synthesizing an identical RNA strand (via a complementary intermediate strand).

According to the invention, the term “template RNA” refers to RNA that can be transcribed or replicated by an RNA-dependent RNA polymerase.

In an embodiment of the invention, the RNA used according to the invention is replicon RNA or simply “a replicon”, in particular self-replicating RNA. In one particularly preferred embodiment, the replicon or self-replicating RNA is derived from or comprises elements derived from a ssRNA virus, in particular a positive-stranded ssRNA virus such as an alphavirus.

In general, RNA viruses are a diverse group of infectious particles with an RNA genome. RNA viruses can be sub-grouped into single stranded RNA (ssRNA) and double-stranded RNA (dsRNA) viruses, and the ssRNA viruses can be further generally divided into positive-stranded [(+) stranded] and/or negative-stranded [(−) stranded] viruses. Positive-stranded RNA viruses are prima facie attractive as a delivery system in biomedicine because their RNA may serve directly as template for translation in the host cell.

Alphaviruses are typical representatives of positive-stranded RNA viruses. The hosts of alphaviruses include a wide range of organisms, comprising insects, fish and mammals, such as domesticated animals and humans. Alphaviruses replicate in the cytoplasm of infected cells (for review of the alphaviral life cycle see Jose et al., 2009, Future Microbiol. 4:837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP 1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1.

In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res. 87:111-124). Following infection, i.e., at early stages of the viral life cycle, the (+) stranded genomic RNA directly acts like a messenger RNA for the translation of the open reading frame encoding the non-structural poly-protein (nsP1234). In some alphaviruses, there is an opal stop codon between the coding sequences of nsP3 and nsP4: polyprotein P123, containing nsP1, nsP2, and nsP3, is produced when translation terminates at the opal stop codon, and polyprotein P1234, containing in addition nsP4, is produced upon readthrough of this opal codon (Strauss & Strauss, 1994, Microbiol. Rev. 58:491-562; Rupp et al., 2015, J. Gen. Virology 96:2483-2500). nsP1234 is autoproteolytically cleaved into the fragments nsP123 and nsP4. The polypeptides nsP123 and nsP4 associate to form the (−) strand replicase complex that transcribes (−) stranded RNA, using the (+) stranded genomic RNA as template. Typically at later stages, the nsP123 fragment is completely cleaved into individual proteins nsP1, nsP2 and nsP3 (Shirako & Strauss, 1994, J. Virol. 68:1874-1885). All four proteins form the (+) strand replicase complex that synthesizes new (+) stranded genomes, using the (−) stranded complement of genomic RNA as template (Kim et al., 2004, Virology 323:153-163, Vasiljeva et al., 2003, J. Biol. Chem. 278:41636-41645).

In infected cells, subgenomic RNA as well as new genomic RNA is provided with a 5′-cap by nsP1 (Pettersson et al. 1980, Eur. J. Biochem. 105:435-443; Rozanov et al., 1992, J. Gen. Virology 73:2129-2134), and provided with a poly-adenylate [poly(A)] tail by nsP4 (Rubach et al., 2009, Virology 384:201-208). Thus, both subgenomic RNA and genomic RNA resemble messenger RNA (mRNA).

Alphavirus structural proteins are typically encoded by one single open reading frame under control of a subgenomic promoter (Strauss & Strauss, 1994, Microbiol. Rev. 58:491-562). The subgenomic promoter is recognized by alphaviral non-structural proteins acting in cis. In particular, alphavirus replicase synthesizes a (+) stranded subgenomic transcript using the (−) stranded complement of genomic RNA as template. The (+) stranded subgenomic transcript encodes the alphavirus structural proteins (Kim et al., 2004, Virology 323:153-163, Vasiljeva et al., 2003, J. Biol. Chem. 278:41636-41645). The subgenomic RNA transcript serves as template for translation of the open reading frame encoding the structural proteins as one poly-protein, and the poly-protein is cleaved to yield the structural proteins. At a late stage of alphavirus infection in a host cell, a packaging signal which is located within the coding sequence of nsP2 ensures selective packaging of genomic RNA into budding virions, packaged by structural proteins (White et al., 1998, J. Virol. 72:4320-4326).

In infected cells, (−) strand RNA synthesis is typically observed only in the first 3-4 h post infection, and is undetectable at late stages, at which time the synthesis of only (+) strand RNA (both genomic and subgenomic) is observed. According to Frolov et al., 2001, RNA 7:1638-1651, the prevailing model for regulation of RNA synthesis suggests a dependence on the processing of the non-structural poly-protein: initial cleavage of the non-structural polyprotein nsP1234 yields nsP123 and nsP4; nsP4 acts as RNA-dependent RNA polymerase (RdRp) that is active for (−) strand synthesis, but inefficient for the generation of (+) strand RNAs. Further processing of the polyprotein nsP123, including cleavage at the nsP2/nsP3 junction, changes the template specificity of the replicase to increase synthesis of (+) strand RNA and to decrease or terminate synthesis of (−) strand RNA.

The synthesis of alphaviral RNA is also regulated by cis-acting RNA elements, including four conserved sequence elements (CSEs; Strauss & Strauss, 1994, Microbiol. Rev. 58:491-562; and Frolov, 2001, RNA 7:1638-1651).

In general, the 5′ replication recognition sequence of the alphavirus genome is characterized by low overall homology between different alphaviruses, but has a conserved predicted secondary structure. The 5′ replication recognition sequence of the alphavirus genome is not only involved in translation initiation, but also comprises the 5′ replication recognition sequence comprising two conserved sequence elements involved in synthesis of viral RNA, CSE 1 and CSE 2. For the function of CSE 1 and 2, the secondary structure is believed to be more important than the linear sequence (Strauss & Strauss, 1994, Microbiol. Rev. 58:491-562).

In contrast, the 3′ terminal sequence of the alphavirus genome, i.e., the sequence immediately upstream of the poly(A) sequence, is characterized by a conserved primary structure, particularly by conserved sequence element 4 (CSE 4), also termed “19-nt conserved sequence”, which is important for initiation of (−) strand synthesis.

CSE 3, also termed “junction sequence” is a conserved sequence element on the (+) strand of alphaviral genomic RNA, and the complement of CSE 3 on the (−) strand acts as promoter for subgenomic RNA transcription (Strauss & Strauss, 1994, Microbiol. Rev. 58:491-562; Frolov et al., 2001, RNA 7:1638-1651). CSE 3 typically overlaps with the region encoding the C-terminal fragment of nsP4.

In addition to alphavirus proteins, also host cell factors, presumably proteins, may bind to conserved sequence elements (Strauss & Strauss, supra).

Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase (typically as poly-protein nsP1234), and the other nucleic acid molecule is capable of being replicated by said replicase in trans (hence the designation trans-replication system). trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

According to the invention, the term “alphavirus” is to be understood broadly and includes any virus particle that has characteristics of alphaviruses. Characteristics of alphavirus include the presence of a (+) stranded RNA which encodes genetic information suitable for replication in a host cell, including RNA polymerase activity. Further characteristics of many alphaviruses are described e.g., in Strauss & Strauss, 1994, Microbiol. Rev. 58:491-562. The term “alphavirus” includes alphavirus found in nature, as well as any variant or derivative thereof. In some embodiments, a variant or derivative is not found in nature.

In one embodiment, the alphavirus is an alphavirus found in nature. Typically, an alphavirus found in nature is infectious to any one or more eukaryotic organisms, such as an animal (including a vertebrate such as a human, and an arthropod such as an insect).

An alphavirus found in nature is preferably selected from the group consisting of the following: Barmah Forest virus complex (comprising Barmah Forest virus); Eastern equine encephalitis complex (comprising seven antigenic types of Eastern equine encephalitis virus); Middelburg virus complex (comprising Middelburg virus); Ndumu virus complex (comprising Ndumu virus); Semliki Forest virus complex (comprising Bebaru virus, Chikungunya virus, Mayaro virus and its subtype Una virus, O'Nyong Nyong virus, and its subtype Igbo-Ora virus, Ross River virus and its subtypes Bebaru virus, Getah virus, Sagiyama virus, Semliki Forest virus and its subtype Me Tri virus); Venezuelan equine encephalitis complex (comprising Cabassou virus, Everglades virus, Mosso das Pedras virus, Mucambo virus, Paramana virus, Pixuna virus, Rio Negro virus, Trocara virus and its subtype Bijou Bridge virus, Venezuelan equine encephalitis virus); Western equine encephalitis complex (comprising Aura virus, Babanki virus, Kyzylagach virus, Sindbis virus, Ockelbo virus, Whataroa virus, Buggy Creek virus, Fort Morgan virus, Highlands J virus, Western equine encephalitis virus); and some unclassified viruses including Salmon pancreatic disease virus; Sleeping Disease virus; Southern elephant seal virus; Tonate virus. More preferably, the alphavirus is selected from the group consisting of Semliki Forest virus complex (comprising the virus types as indicated above, including Semliki Forest virus), Western equine encephalitis complex (comprising the virus types as indicated above, including Sindbis virus), Eastern equine encephalitis virus (comprising the virus types as indicated above), Venezuelan equine encephalitis complex (comprising the virus types as indicated above, including Venezuelan equine encephalitis virus).

In a further preferred embodiment, the alphavirus is Semliki Forest virus. In an alternative further preferred embodiment, the alphavirus is Sindbis virus. In an alternative further preferred embodiment, the alphavirus is Venezuelan equine encephalitis virus.

In some embodiments of the present invention, the alphavirus is not an alphavirus found in nature. Typically, an alphavirus not found in nature is a variant or derivative of an alphavirus found in nature, that is distinguished from an alphavirus found in nature by at least one mutation in the nucleotide sequence, i.e., the genomic RNA. The mutation in the nucleotide sequence may be selected from an insertion, a substitution or a deletion of one or more nucleotides, compared to an alphavirus found in nature. A mutation in the nucleotide sequence may or may not be associated with a mutation in a polypeptide or protein encoded by the nucleotide sequence. For example, an alphavirus not found in nature may be an attenuated alphavirus. An attenuated alphavirus not found in nature is an alphavirus that typically has at least one mutation in its nucleotide sequence by which it is distinguished from an alphavirus found in nature, and that is either not infectious at all, or that is infectious but has a lower disease-producing ability or no disease-producing ability at all. As an illustrative example, TC83 is an attenuated alphavirus that is distinguished from the Venezuelan equine encephalitis virus (VEEV) found in nature (McKinney et al., 1963, Am. J. Trop. Med. Hyg. 12:597-603).

Members of the alphavirus genus may also be classified based on their relative clinical features in humans: alphaviruses associated primarily with encephalitis, and alphaviruses associated primarily with fever, rash, and polyarthritis.

The term “alphaviral” means found in an alphavirus, or originating from an alphavirus or derived from an alphavirus, e.g., by genetic engineering.

According to the invention, “SFV” stands for Semliki Forest virus. According to the invention, “SIN” or “SINV” stands for Sindbis virus. According to the invention, “VEE” or “VEEV” stands for Venezuelan equine encephalitis virus.

According to the invention, the term “of an alphavirus” or “derived from an alphavirus” refers to an entity of origin from an alphavirus. For illustration, a protein of an alphavirus may refer to a protein that is found in alphavirus and/or to a protein that is encoded by alphavirus; and a nucleic acid sequence of an alphavirus may refer to a nucleic acid sequence that is found in alphavirus and/or to a nucleic acid sequence that is encoded by alphavirus. Preferably, a nucleic acid sequence “of an alphavirus” refers to a nucleic acid sequence “of the genome of an alphavirus” and/or “of genomic RNA of an alphavirus”.

According to the invention, the term “alphaviral RNA” refers to any one or more of alphaviral genomic RNA (i.e., (+) strand), complement of alphaviral genomic RNA (i.e., (−) strand), and the subgenomic transcript (i.e., (+) strand), or a fragment of any thereof.

According to the invention, “alphavirus genome” refers to genomic (+) strand RNA of an alphavirus.

According to the invention, the term “native alphavirus sequence” and similar terms typically refer to a (e.g., nucleic acid) sequence of a naturally occurring alphavirus (alphavirus found in nature). In some embodiments, the term “native alphavirus sequence” also includes a sequence of an attenuated alphavirus.

According to the invention, the term “5′ replication recognition sequence” preferably refers to a continuous nucleic acid sequence, preferably a ribonucleic acid sequence, that is identical or homologous to a 5′ fragment of the alphavirus genome. The “5′ replication recognition sequence” is a nucleic acid sequence that can be recognized by an alphaviral replicase. The term 5′ replication recognition sequence includes native 5′ replication recognition sequences as well as functional equivalents thereof, such as, e.g., functional variants of a 5′ replication recognition sequence of alphavirus found in nature. The 5′ replication recognition sequence is required for synthesis of the (−) strand complement of alphavirus genomic RNA, and is required for synthesis of (+) strand viral genomic RNA based on a (−) strand template. A native 5′ replication recognition sequence typically encodes at least the N-terminal fragment of nsP1; but does not comprise the entire open reading frame encoding nsP1234. In view of the fact that a native 5′ replication recognition sequence typically encodes at least the N-terminal fragment of nsP1, a native 5′ replication recognition sequence typically comprises at least one initiation codon, typically AUG. In one embodiment, the 5′ replication recognition sequence comprises conserved sequence element 1 of an alphavirus genome (CSE 1) or a variant thereof and conserved sequence element 2 of an alphavirus genome (CSE 2) or a variant thereof. The 5′ replication recognition sequence is typically capable of forming four stem loops (SL), i.e., SL1, SL2, SL3, SL4. The numbering of these stem loops begins at the 5′ end of the 5′ replication recognition sequence.

According to the invention, the term “3′ replication recognition sequence” preferably refers to a continuous nucleic acid sequence, preferably a ribonucleic acid sequence, that is identical or homologous to a 3′ fragment of the alphavirus genome. The “3′ replication recognition sequence” is a nucleic acid sequence that can be recognized by an alphaviral replicase. The term 3′ replication recognition sequence includes native 3′ replication recognition sequences as well as functional equivalents thereof, such as, e.g., functional variants of a 3′ replication recognition sequence of alphavirus found in nature. The 3′ replication recognition sequence is required for synthesis of the (−) strand complement of alphavirus genomic RNA. In one embodiment, the 3′ replication recognition sequence comprises conserved sequence element 4 of an alphavirus genome (CSE 4) or a variant thereof and optionally the poly(A) tail of an alphavirus genome.

The term “conserved sequence element” or “CSE” refers to a nucleotide sequence found in alphavirus RNA. These sequence elements are termed “conserved” because orthologs are present in the genome of different alphaviruses, and orthologous CSEs of different alphaviruses preferably share a high percentage of sequence identity and/or a similar secondary or tertiary structure. The term CSE includes CSE 1, CSE 2, CSE 3 and CSE 4.

According to the invention, the terms “CSE 1” or “44-nt CSE” synonymously refer to a nucleotide sequence that is required for (+) strand synthesis from a (−) strand template. The term “CSE 1” refers to a sequence on the (+) strand; and the complementary sequence of CSE 1 (on the (−) strand) functions as a promoter for (+) strand synthesis. Preferably, the term CSE 1 includes the most 5′ nucleotide of the alphavirus genome. CSE 1 typically forms a conserved stem-loop structure. Without wishing to be bound to a particular theory, it is believed that, for CSE 1, the secondary structure is more important than the primary structure, i.e., the linear sequence. In genomic RNA of the model alphavirus Sindbis virus, CSE 1 consists of a consecutive sequence of 44 nucleotides, which is formed by the most 5′ 44 nucleotides of the genomic RNA (Strauss & Strauss, 1994, Microbiol. Rev. 58:491-562).

According to the invention, the terms “CSE 2” or “51-nt CSE” synonymously refer to a nucleotide sequence that is required for (−) strand synthesis from a (+) strand template. The (+) strand template is typically alphavirus genomic RNA or an RNA replicon (note that the subgenomic RNA transcript, which does not comprise CSE 2, does not function as a template for (−) strand synthesis). In alphavirus genomic RNA, CSE 2 is typically localized within the coding sequence for nsP 1. In genomic RNA of the model alphavirus Sindbis virus, the 51-nt CSE is located at nucleotide positions 155-205 of genomic RNA (Frolov et al., 2001, RNA 7:1638-1651). CSE 2 forms typically two conserved stem loop structures. These stem loop structures are designated as stem loop 3 (SL3) and stem loop 4 (SL4) because they are the third and fourth conserved stem loop, respectively, of alphavirus genomic RNA, counted from the 5′ end of alphavirus genomic RNA. Without wishing to be bound to a particular theory, it is believed that, for CSE 2, the secondary structure is more important than the primary structure, i.e., the linear sequence.

According to the invention, the terms “CSE 3” or “junction sequence” synonymously refer to a nucleotide sequence that is derived from alphaviral genomic RNA and that comprises the start site of the subgenomic RNA. The complement of this sequence in the (−) strand acts to promote subgenomic RNA transcription. In alphavirus genomic RNA, CSE 3 typically overlaps with the region encoding the C-terminal fragment of nsP4 and extends to a short non-coding region located upstream of the open reading frame encoding the structural proteins.

According to the invention, the terms “CSE 4” or “19-nt conserved sequence” or “19-nt CSE” synonymously refer to a nucleotide sequence from alphaviral genomic RNA, immediately upstream of the poly(A) sequence in the 3′ untranslated region of the alphavirus genome. CSE 4 typically consists of 19 consecutive nucleotides. Without wishing to be bound to a particular theory, CSE 4 is understood to function as a core promoter for initiation of (−) strand synthesis (Jose et al., 2009, Future Microbiol. 4:837-856); and/or CSE 4 and the poly(A) tail of the alphavirus genomic RNA are understood to function together for efficient (−) strand synthesis (Hardy & Rice, 2005, J. Virol. 79:4630-4639).

According to the invention, the term “subgenomic promoter” or “SGP” refers to a nucleic acid sequence upstream (5′) of a nucleic acid sequence (e.g., coding sequence), which controls transcription of said nucleic acid sequence by providing a recognition and binding site for RNA polymerase, typically RNA-dependent RNA polymerase, in particular functional alphavirus non-structural protein. The SGP may include further recognition or binding sites for further factors. A subgenomic promoter is typically a genetic element of a positive strand RNA virus, such as an alphavirus. A subgenomic promoter of alphavirus is a nucleic acid sequence comprised in the viral genomic RNA. The subgenomic promoter is generally characterized in that it allows initiation of the transcription (RNA synthesis) in the presence of an RNA-dependent RNA polymerase, e.g., functional alphavirus non-structural protein. AN RNA (−) strand, i.e., the complement of alphaviral genomic RNA, serves as a template for synthesis of a (+) strand subgenomic transcript, and synthesis of the (+) strand subgenomic transcript is typically initiated at or near the subgenomic promoter. The term “subgenomic promoter” as used herein, is not confined to any particular localization in a nucleic acid comprising such subgenomic promoter. In some embodiments, the SGP is identical to CSE 3 or overlaps with CSE 3 or comprises CSE 3.

The terms “subgenomic transcript” or “subgenomic RNA” synonymously refer to an RNA molecule that is obtainable as a result of transcription using an RNA molecule as template (“template RNA”), wherein the template RNA comprises a subgenomic promoter that controls transcription of the subgenomic transcript. The subgenomic transcript is obtainable in the presence of an RNA-dependent RNA polymerase, in particular functional alphavirus non-structural protein. For instance, the term “subgenomic transcript” may refer to the RNA transcript that is prepared in a cell infected by an alphavirus, using the (−) strand complement of alphavirus genomic RNA as template. However, the term “subgenomic transcript”, as used herein, is not limited thereto and also includes transcripts obtainable by using heterologous RNA as template. For example, subgenomic transcripts are also obtainable by using the (−) strand complement of SGP-containing replicons according to the present invention as template. Thus, the term “subgenomic transcript” may refer to an RNA molecule that is obtainable by transcribing a fragment of alphavirus genomic RNA, as well as to an RNA molecule that is obtainable by transcribing a fragment of a replicon according to the present invention.

According to the invention, a nucleic acid construct that is capable of being replicated by a replicase, preferably an alphaviral replicase, is termed replicon. According to the invention, the term “replicon” defines an RNA molecule that can be replicated by RNA-dependent RNA polymerase, yielding—without DNA intermediate—one or multiple identical or essentially identical copies of the RNA replicon. “Without DNA intermediate” means that no deoxyribonucleic acid (DNA) copy or complement of the replicon is formed in the process of forming the copies of the RNA replicon, and/or that no deoxyribonucleic acid (DNA) molecule is used as a template in the process of forming the copies of the RNA replicon, or complement thereof. The replicase function is typically provided by functional alphavirus non-structural protein.

According to the invention, the terms “can be replicated” and “capable of being replicated” generally describe that one or more identical or essentially identical copies of a nucleic acid can be prepared. When used together with the term “replicase”, such as in “capable of being replicated by a replicase”, the terms “can be replicated” and “capable of being replicated” describe functional characteristics of a nucleic acid molecule, e.g., an RNA replicon, with respect to a replicase. These functional characteristics comprise at least one of (i) the replicase is capable of recognizing the replicon and (ii) the replicase is capable of acting as RNA-dependent RNA polymerase (RdRP). Preferably, the replicase is capable of both (i) recognizing the replicon and (ii) acting as RNA-dependent RNA polymerase.

The expression “capable of recognizing” describes that the replicase is capable of physically associating with the replicon, and preferably, that the replicase is capable of binding to the replicon, typically non-covalently. The term “binding” can mean that the replicase has the capacity of binding to any one or more of a conserved sequence element 1 (CSE 1) or complementary sequence thereof (if comprised by the replicon), conserved sequence element 2 (CSE 2) or complementary sequence thereof (if comprised by the replicon), conserved sequence element 3 (CSE 3) or complementary sequence thereof (if comprised by the replicon), conserved sequence element 4 (CSE 4) or complementary sequence thereof (if comprised by the replicon). Preferably, the replicase is capable of binding to CSE 2 [i.e., to the (+) strand] and/or to CSE 4 [i.e., to the (+) strand], or of binding to the complement of CSE 1 [i.e., to the (−) strand] and/or to the complement of CSE 3 [i.e., to the (−) strand].

The expression “capable of acting as RdRP” means that the replicase is capable to catalyze the synthesis of the (−) strand complement of alphaviral genomic (+) strand RNA, wherein the (+) strand RNA has template function, and/or that the replicase is capable to catalyze the synthesis of (+) strand alphaviral genomic RNA, wherein the (−) strand RNA has template function. In general, the expression “capable of acting as RdRP” can also include that the replicase is capable to catalyze the synthesis of a (+) strand subgenomic transcript wherein a (−) strand RNA has template function, and wherein synthesis of the (+) strand subgenomic transcript is typically initiated at an alphavirus subgenomic promoter.

The expressions “capable of binding” and “capable of acting as RdRP” refer to the capability at normal physiological conditions. In particular, they refer to the conditions inside a cell, which expresses functional alphavirus non-structural protein or which has been transfected with a nucleic acid that codes for functional alphavirus non-structural protein. The cell is preferably a eukaryotic cell. The capability of binding and/or the capability of acting as RdRP can be experimentally tested, e.g., in a cell-free in vitro system or in a eukaryotic cell. Optionally, said eukaryotic cell is a cell from a species to which the particular alphavirus that represents the origin of the replicase is infectious. For example, when the alphavirus replicase from a particular alphavirus is used that is infectious to humans, the normal physiological conditions are conditions in a human cell. More preferably, the eukaryotic cell (in one example human cell) is from the same tissue or organ to which the particular alphavirus that represents the origin of the replicase is infectious.

According to the invention, “compared to a native alphavirus sequence” and similar terms refer to a sequence that is a variant of a native alphavirus sequence. The variant is typically not itself a native alphavirus sequence.

In one embodiment, the RNA replicon comprises a replication recognition sequence such as a 5′ replication recognition sequence and a 3′ replication recognition sequence. A replication recognition sequence is a nucleic acid sequence that can be recognized by functional alphavirus non-structural protein. In other words, functional alphavirus non-structural protein is capable of recognizing the replication recognition sequence. Preferably, the 5′ replication recognition sequence is located at the 5′ end of the replicon. In one embodiment, the 5′ replication recognition sequence consists of or comprises CSE 1 and 2. Preferably, the 3′ replication recognition sequence is located at the 3′ end of the replicon (if the replicon does not comprise a poly(A) tail), or immediately upstream of the poly(A) tail (if the replicon comprises a poly(A) tail). In one embodiment, the 3′ replication recognition sequence consists of or comprises CSE 4.

In one embodiment, the 5′ replication recognition sequence and the 3′ replication recognition sequence are capable of directing replication of the RNA replicon in the presence of functional alphavirus non-structural protein. Thus, when present alone or preferably together, these recognition sequences direct replication of the RNA replicon in the presence of functional alphavirus non-structural protein.

It is preferable that a functional alphavirus non-structural protein is provided in cis (encoded as protein of interest by an open reading frame on the replicon) or in trans (encoded as protein of interest by an open reading frame on a separate replicase construct, that is capable of recognizing both the 5′ replication recognition sequence and the 3′ replication recognition sequence of the replicon. In one embodiment, this is achieved when the 5′ and 3′ replication recognition sequences are native to the alphavirus from which the functional alphavirus non-structural protein is derived. Native means that the natural origin of these sequences is the same alphavirus. In an alternative embodiment, the 5′ replication recognition sequence and/or the 3′ replication recognition sequence are not native to the alphavirus from which the functional alphavirus non-structural protein is derived, provided that the functional alphavirus non-structural protein is capable of recognizing both the 5′ replication recognition sequence and the 3′ replication recognition sequence of the replicon. In other words, the functional alphavirus non-structural protein is compatible to the 5′ replication recognition sequence and the 3′ replication recognition sequence. When a non-native functional alphavirus non-structural protein is capable of recognizing a respective sequence or sequence element, the functional alphavirus non-structural protein is said to be compatible (cross-virus compatibility). Any combination of (3′/5′) replication recognition sequences and CSEs, respectively, with functional alphavirus non-structural protein is possible as long as cross-virus compatibility exists. Cross-virus compatibility can readily be tested by the skilled person working the present invention by incubating a functional alphavirus non-structural protein to be tested together with an RNA, wherein the RNA has 3′- and 5′ replication recognition sequences to be tested, at conditions suitable for RNA replication, e.g., in a suitable host cell. If replication occurs, the (3′/5′) replication recognition sequences and the functional alphavirus non-structural protein are determined to be compatible.

In one embodiment of the invention, the replicon is part of a trans-replication system and, thus, the replicon is a trans-replicon. In this embodiment, it is preferred that the RNA replicon does not comprise an open reading frame encoding functional alphavirus non-structural protein. Thus, in this embodiment, the present invention provides a system comprising two nucleic acid molecules: a first RNA construct for expressing functional alphavirus non-structural protein (i.e., encoding functional alphavirus non-structural protein); and a second RNA molecule, the RNA replicon. The RNA construct for expressing functional alphavirus non-structural protein is synonymously referred to herein as “RNA construct for expressing functional alphavirus non-structural protein” or as “replicase construct”. The functional alphavirus non-structural protein is as defined above and is typically encoded by an open reading frame comprised by the replicase construct. The functional alphavirus non-structural protein encoded by the replicase construct may be any functional alphavirus non-structural protein that is capable of replicating the replicon. According to the invention, the replicase construct may be present with the replicon(s) within the same composition, e.g., as mixed particulate formulation or combined particulate formulation, or in separate compositions, e.g., as individual particulate formulations. When the system of the present invention is introduced into a cell, preferably a eukaryotic cell, the open reading frame encoding functional alphavirus non-structural protein can be translated. After translation, the functional alphavirus non-structural protein is capable of replicating a separate RNA molecule (RNA replicon) in trans.

Herein, trans (e.g., in the context of trans-acting, trans-regulatory), in general, means “acting from a different molecule” (i.e., intermolecular). It is the opposite of cis (e.g., in the context of cis-acting, cis-regulatory), which, in general, means “acting from the same molecule” (i.e., intramolecular). In the context of RNA synthesis (including transcription and RNA replication), a trans-acting element includes a nucleic acid sequence that contains a gene encoding an enzyme capable of RNA synthesis (RNA polymerase). The RNA polymerase uses a second nucleic acid molecule, i.e., a nucleic acid molecule other than the one by which it is encoded, as template for the synthesis of RNA. Both the RNA polymerase and the nucleic acid sequence that contains a gene encoding the RNA polymerase are said to “act in trans” on the second nucleic acid molecule. In the context of the present invention, the RNA polymerase encoded by the trans-acting RNA may be functional alphavirus non-structural protein. The functional alphavirus non-structural protein is capable of using a second nucleic acid molecule, which is an RNA replicon, as template for the synthesis or RNA, including replication of the RNA replicon. The RNA replicon that can be replicated by the replicase in trans according to the present invention is synonymously referred to herein as “trans-replicon”.

According to the present invention, the role of the functional alphavirus non-structural protein is to amplify the replicon, and to prepare a subgenomic transcript, if a subgenomic promoter is present on the replicon. If the replicon encodes a gene of interest for expression, the expression levels of the gene of interest and/or the duration of expression may be regulated in trans by modifying the levels of the functional alphavirus non-structural protein.

The trans-replication system of the present invention comprises at least two nucleic acid molecules. In a preferred embodiment, the system consists of exactly two RNA molecules, the replicon and the replicase construct. In alternative preferred embodiments, the system comprises more than one replicon, each preferably encoding at least one protein of interest, and also comprises the replicase construct. In these embodiments, the functional alphavirus non-structural protein encoded by the replicase construct can act on each replicon to drive replication and optionally production of subgenomic transcripts, respectively. For example, each replicon may encode a pharmaceutically active peptide or protein.

Preferably, the replicase construct lacks at least one conserved sequence element (CSE) that is required for (−) strand synthesis based on a (+) strand template, and/or for (+) strand synthesis based on a (−) strand template. More preferably, the replicase construct does not comprise any alphaviral conserved sequence elements (CSEs). In particular, among the four CSEs of alphavirus (Strauss & Strauss, 1994, Microbiol. Rev. 58:491-562; Jose et al., 2009, Future Microbiol. 4:837-856), any one or more of the following CSEs are preferably not present on the replicase construct: CSE 1; CSE 2; CSE 3; CSE 4. Particularly in the absence of any one or more alphaviral CSE, the replicase construct of the present invention resembles typical eukaryotic mRNA much more than it resembles alphaviral genomic RNA.

The replicase construct of the present invention is preferably distinguished from alphaviral genomic RNA at least in that it is not capable of self-replication and/or that it does not comprise an open reading frame under the control of a sub-genomic promoter. When unable to self-replicate, the replicase construct may also be termed “suicide construct”.

The replicase construct according to the present invention is preferably a single stranded RNA molecule. The replicase construct according to the present invention is typically a (+) stranded RNA molecule. In one embodiment, the replicase construct of the present invention is an isolated nucleic acid molecule.

In one embodiment, the RNA such as the replicon according to the present invention comprises at least one open reading frame encoding an agent, e.g., a peptide or polypeptide, of interest. In various embodiments, the agent of interest is encoded by a heterologous nucleic acid sequence. According to the present invention, the term “heterologous” refers to the fact that a nucleic acid sequence is not naturally functionally or structurally linked to a nucleic sequence such as an alphavirus nucleic acid sequence.

The RNA according to the present invention may encode a single agent or multiple agents. For example, multiple polypeptides can be encoded as a single polypeptide (fusion polypeptide) or as separate polypeptides. In some embodiments, the RNA according to the present invention may comprise more than one open reading frame, each of which in the case of a replicon may independently be selected to be under the control of a subgenomic promoter or not. Alternatively, a poly-protein or fusion polypeptide comprises individual polypeptides separated by an optionally autocatalytic protease cleavage site (e.g., foot-and-mouth disease virus 2A protein), or an intein.

Nucleic acids can be transferred into a host cell by physical, chemical or biological means. Physical methods for introducing a nucleic acid into a host cell include calcium phosphate precipitation, lipofection, particle bombardment, microinjection, electroporation, and the like. Biological methods for introducing a nucleic acid of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. Chemical means for introducing a nucleic acid into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

According to the invention it is preferred that a nucleic acid such as RNA encoding an agent, e.g., a peptide or polypeptide, once taken up by or introduced, i.e., transfected or transduced, into a cell which cell may be present in vitro or in a subject results in expression of said agent. The cell may express the encoded agent intracellularly (e.g., in the cytoplasm and/or in the nucleus), may secrete the encoded agent, or may express it on the surface.

According to the invention, terms such as “nucleic acid expressing” and “nucleic acid encoding” or similar terms are used interchangeably herein and with respect to a particular agent mean that the nucleic acid, if present in the appropriate environment, preferably within a cell, can be expressed to produce said agent.

Terms such as “transferring”, “introducing”, “transfecting” or “transducing” are used interchangeably herein and relate to the introduction of nucleic acids, in particular exogenous or heterologous nucleic acids, such as RNA into a cell. According to the present invention, the cell can be present in vitro or in vivo, e.g., the cell can form part of an organ, a tissue and/or an organism. According to the invention, transfection can be transient or stable. For some applications of transfection, it is sufficient if the transfected genetic material is only transiently expressed. Since the nucleic acid introduced in the transfection process is usually not integrated into the nuclear genome, the foreign nucleic acid will be diluted through mitosis or degraded. Cell lines allowing episomal amplification of nucleic acids greatly reduce the rate of dilution.

If it is desired that the transfected nucleic acid actually remains in the genome of the cell and its daughter cells, a stable transfection must occur. RNA can be transfected into cells to transiently express its encoded agent, e.g., protein.

According to the present invention, the term “peptide” refers to substances comprising two or more, preferably 3 or more, preferably 4 or more, preferably 6 or more, preferably 8 or more, preferably 10 or more, preferably 13 or more, preferably 16 or more, preferably 21 or more and up to preferably 8, 10, 20, 30, 40 or 50, in particular 100 amino acids joined covalently by peptide bonds.

The term “protein” refers to large peptides, i.e., polypeptides, preferably to peptides with more than 100 amino acid residues, but in general the terms “peptide”, “polypeptide” and “protein” are synonyms and are used interchangeably herein.

The terms “peptide” and “polypeptide” comprise, according to the invention, substances which contain not only amino acid components but also non-amino acid components such as sugars and phosphate structures, and also comprise substances containing bonds such as ester, thioether or disulfide bonds.

The present invention also includes “variants” of the nucleic acids, peptides, proteins, or amino acid sequences described herein, such as naturally occurring sequences. The term “variant” with respect to, for example, nucleic acid and amino acid sequences, according to the invention includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code (e.g., due to adaption of the codon usage). A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence.

According to the invention, nucleic acid variants include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

“Fragment”, with reference to a nucleic acid sequence, relates to a part of a nucleic acid sequence, i.e., a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present invention those fragments of RNA molecules are preferred which retain RNA stability and/or translational efficiency.

According to the invention, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the invention, a nucleic acid variant can comprise one or more nucleotide changes in comparison with the reference nucleic acid.

Variants of specific nucleic acid sequences preferably have at least one functional property of said specific sequences and preferably are functionally equivalent to said specific sequences, e.g., nucleic acid sequences exhibiting properties identical or similar to those of the specific nucleic acid sequences.

Preferably the degree of identity between a given nucleic acid sequence and a nucleic acid sequence which is a variant of said given nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

For the purposes of the present invention, “variants” of an amino acid sequence comprise amino acid insertion variants, amino acid addition variants, amino acid deletion variants and/or amino acid substitution variants. Variants of specific amino acid sequences preferably have at least one functional property of said specific sequences and preferably are functionally equivalent to said specific sequences, e.g., amino acid sequences exhibiting properties identical or similar to those of the specific amino acid sequences. In one embodiment, the functional property is a cytotoxic property.

“Fragment”, with reference to an amino acid sequence (peptide or protein), relates to a part of an amino acid sequence, i.e., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants.

Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

Preferably the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. The degree of similarity or identity is given preferably for a segment of at least 80, at least 100, at least 120, at least 150, at least 180, at least 200 or at least 250 amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “% identity” is intended to refer, in particular, to a percentage of amino acid residues which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, and with the aid of the similarity search algorithm by Pearson and Lipman, 1988, Proc. Natl Acad. Sci. USA 85, 2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

Homologous amino acid sequences exhibit according to the invention at least 40%, in particular at least 50%, at least 60%, at least 70%, at least 80%, at least 90% and preferably at least 95%, at least 98 or at least 99% identity of the amino acid residues.

Derivatives of the peptides or proteins described herein are comprised by the terms “peptide” and “protein”. “Derivatives” of proteins and peptides are modified forms of proteins and peptides. Such modifications include any chemical modification and comprise single or multiple substitutions, deletions and/or additions of any molecules associated with the protein or peptide, such as carbohydrates, lipids and/or proteins or peptides. In one embodiment, “derivatives” of proteins or peptides include those modified analogs resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “derivative” also extends to all functional chemical equivalents of said proteins and peptides. Preferably, a modified peptide has increased stability and/or increased activity.

The term “derived” means according to the invention that a particular entity, in particular a particular sequence, is present in the object from which it is derived, in particular an organism or molecule. In the case of amino acid or nucleic acid sequences, especially particular sequence regions, “derived” in particular means that the relevant amino acid sequence or nucleic acid sequence is derived from an amino acid sequence or nucleic acid sequence in which it is present.

The term “cell” or “host cell” preferably is an intact cell, i.e., a cell with an intact membrane that has not released its normal intracellular components such as enzymes, organelles, or genetic material. An intact cell preferably is a viable cell, i.e., a living cell capable of carrying out its normal metabolic functions. Preferably said term relates according to the invention to any cell which can be transformed or transfected with an exogenous nucleic acid. The term “cell” includes according to the invention prokaryotic cells (e.g., E. coli) or eukaryotic cells (e.g., dendritic cells, B cells, epithelial cells, CHO cells, COS cells, K562 cells, HEK293 cells, HELA cells, yeast cells, and insect cells). The exogenous nucleic acid may be found inside the cell (i) freely dispersed as such, (ii) incorporated in a recombinant vector, or (iii) integrated into the host cell genome or mitochondrial DNA. Mammalian cells are particularly preferred, such as cells from humans, mice, hamsters, pigs, goats, and primates. The cells may be derived from a large number of tissue types and include primary cells and cell lines.

A cell which comprises a nucleic acid, e.g., which has been transfected with a nucleic acid, preferably expresses the agent, e.g., peptide or polypeptide, encoded by the nucleic acid.

Terms such as “reducing”, “inhibiting” or “decreasing” relate to the ability to cause an overall decrease, preferably of 5% or greater, 10% or greater, 20% or greater, more preferably of 50% or greater, and most preferably of 75% or greater, in the level. These terms include a complete or essentially complete inhibition, i.e., a reduction to zero or essentially to zero.

Terms such as “increasing”, “enhancing”, “promoting”, “stimulating”, or “inducing” relate to the ability to cause an overall increase, preferably of 5% or greater, 10% or greater, 20% or greater, 50% or greater, 75% or greater, 100% or greater, 200% or greater, or 500% or greater, in the level. These terms may relate to an increase, enhancement, promotion, stimulation, or inducement from zero or a non-measurable or non-detectable level to a level of more than zero or a level which is measurable or detectable. Alternatively, these terms may also mean that there was a certain level before an increase, enhancement, promotion, stimulation, or inducement and after the increase, enhancement, promotion, stimulation, or inducement the level is higher.

According to certain embodiments of the methods of the invention, the RNA molecules described herein may be administered in the form of any pharmaceutical composition suitable for intraarterial administration. In one embodiment of the invention, the RNA molecule to be administered is naked, i.e., not complexed with any kind of delivery material or proteins. In one embodiment of the invention, the RNA molecule to be administered is formulated in a delivery vehicle, preferably a vehicle suitable for intraarterial administration. In one embodiment, the delivery vehicle comprises particles. In one embodiment, the delivery vehicle comprises a lipid. In one embodiment, the lipid comprises a cationic lipid. In one embodiment, the lipid forms a complex with and/or encapsulates the RNA molecule. In one embodiment of the invention, the RNA molecule is formulated in liposomes.

The pharmaceutical compositions of the invention are preferably sterile and contain an effective amount of the agents described herein and optionally of further agents as discussed herein to generate the desired reaction or the desired effect.

Pharmaceutical compositions are usually provided in a uniform dosage form and may be prepared in a manner known per se. A pharmaceutical composition may, e.g., be in the form of a solution or suspension.

A pharmaceutical composition may comprise salts, buffer substances, preservatives, carriers, diluents and/or excipients all of which are preferably pharmaceutically acceptable. The term “pharmaceutically acceptable” refers to the non-toxicity of a material which does not interact with the action of the active component of the pharmaceutical composition.

Salts which are not pharmaceutically acceptable may be used for preparing pharmaceutically acceptable salts and are included in the invention. Pharmaceutically acceptable salts of this kind comprise in a non-limiting way those prepared from the following acids: hydrochloric, hydrobromic, sulfuric, nitric, phosphoric, maleic, acetic, salicylic, citric, formic, malonic, succinic acids, and the like. Pharmaceutically acceptable salts may also be prepared as alkali metal salts or alkaline earth metal salts, such as sodium salts, potassium salts or calcium salts.

Suitable buffer substances for use in a pharmaceutical composition include acetic acid in a salt, citric acid in a salt, boric acid in a salt and phosphoric acid in a salt.

Suitable preservatives for use in a pharmaceutical composition include benzalkonium chloride, chlorobutanol, paraben and thimerosal.

An injectable formulation may comprise a pharmaceutically acceptable excipient such as Ringer Lactate.

The term “carrier” refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a patient. Possible carrier substances include, e.g., sterile water, Ringer, Ringer lactate, sterile sodium chloride solution, polyalkylene glycols, hydrogenated naphthalenes and, in particular, biocompatible lactide polymers, lactide/glycolide copolymers or polyoxyethylene/polyoxy-propylene copolymers.

The term “excipient” when used herein is intended to indicate all substances which may be present in a pharmaceutical composition and which are not active ingredients such as, e.g., carriers, binders, lubricants, thickeners, surface active agents, preservatives, emulsifiers, buffers, flavoring agents, or colorants.

In one embodiment, if the pharmaceutical composition comprises nucleic acids, it comprises at least one cationic entity. In general, cationic lipids, cationic polymers and other substances with positive charges may form complexes with negatively charged nucleic acids. It is possible to stabilize the RNA according to the invention by complexation with cationic compounds, preferably polycationic compounds such as for example a cationic or polycationic peptide or protein. In one embodiment, the pharmaceutical composition according to the present invention comprises at least one cationic molecule selected from the group consisting protamine, polyethylene imine, a poly-L-lysine, a poly-L-arginine, a histone or a cationic lipid.

According to the present invention, a cationic lipid is a cationic amphiphilic molecule, e.g., a molecule which comprises at least one hydrophilic and lipophilic moiety. The cationic lipid can be monocationic or polycationic. Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-0-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Cationic lipids also include lipids with a tertiary amine group, including 1,2-dilinoleyloxy-N,N-dimethyl-3-aminopropane (DLinDMA). Cationic lipids are suitable for formulating RNA in lipid formulations as described herein, such as liposomes, emulsions and lipoplexes. Typically positive charges are contributed by at least one cationic lipid and negative charges are contributed by the RNA. In one embodiment, the pharmaceutical composition comprises at least one helper lipid, in addition to a cationic lipid. The helper lipid may be a neutral or an anionic lipid. The helper lipid may be a natural lipid, such as a phospholipid, or an analogue of a natural lipid, or a fully synthetic lipid, or lipid-like molecule, with no similarities with natural lipids. In the case where a pharmaceutical composition includes both a cationic lipid and a helper lipid, the molar ratio of the cationic lipid to the neutral lipid can be appropriately determined in view of stability of the formulation and the like.

In one embodiment, the pharmaceutical composition according to the present invention comprises protamine. According to the invention, protamine is useful as cationic carrier agent. The term “protamine” refers to any of various strongly basic proteins of relatively low molecular weight that are rich in arginine and are found associated especially with DNA in place of somatic histones in the sperm cells of animals such as fish. In particular, the term “protamine” refers to proteins found in fish sperm that are strongly basic, are soluble in water, are not coagulated by heat, and comprise multiple arginine monomers. According to the invention, the term “protamine” as used herein is meant to comprise any protamine amino acid sequence obtained or derived from native or biological sources including fragments thereof and multimeric forms of said amino acid sequence or fragment thereof. Furthermore, the term encompasses (synthesized) polypeptides which are artificial and specifically designed for specific purposes and cannot be isolated from native or biological sources.

The pharmaceutical composition according to the invention can be buffered, (e.g., with an acetate buffer, a citrate buffer, a succinate buffer, a Tris buffer, a phosphate buffer).

In some embodiments, owing to the instability of non-protected RNA, it is advantageous to provide the RNA molecules of the present invention in complexed or encapsulated form. Respective pharmaceutical compositions are provided in the present invention. In particular, in some embodiments, the pharmaceutical composition of the present invention comprises nucleic acid-containing particles, preferably RNA-containing particles. Respective pharmaceutical compositions are referred to as particulate formulations. In particulate formulations according to the present invention, a particle comprises nucleic acid according to the invention and a pharmaceutically acceptable carrier or a pharmaceutically acceptable vehicle that is suitable for delivery of the nucleic acid. The nucleic acid-containing particles may be, for example, in the form of proteinaceous particles or in the form of lipid-containing particles. Suitable proteins or lipids are referred to as particle forming agents. Proteinaceous particles and lipid-containing particles have been described previously to be suitable for delivery of alphaviral RNA in particulate form (e.g., Strauss & Strauss, 1994, Microbiol. Rev. 58:491-562). In particular, alphavirus structural proteins (provided, e.g., by a helper virus) are a suitable carrier for delivery of RNA in the form of proteinaceous particles.

When the system according to the present invention is formulated as a particulate formulation, it is possible that each RNA species (e.g., replicon, replicase construct) is separately formulated as an individual particulate formulation. In that case, each individual particulate formulation will comprise one RNA species. The individual particulate formulations may be present as separate entities, e.g., in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations).

In one embodiment, a pharmaceutical composition according to the invention comprises more than one individual particle formulation. Respective pharmaceutical compositions are referred to as mixed particulate formulations. Mixed particulate formulations according to the invention are obtainable by forming, separately, individual particulate formulations, as described above, followed by a step of mixing of the individual particulate formulations. By the step of mixing, one formulation comprising a mixed population of RNA-containing particles is obtainable (for illustration, e.g., a first population of particles may contain replicon according to the invention, and a second formulation of particles may contain replicase construct according to the invention). Individual particulate populations may be together in one container, comprising a mixed population of individual particulate formulations.

Alternatively, it is possible that all RNA species of the pharmaceutical composition (e.g., replicon, replicase construct, and optional additional species such as RNA encoding a protein suitable for inhibiting IFN) are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

In one embodiment, the particulate formulation of the present invention is a nanoparticulate formulation. In that embodiment, the composition according to the present invention comprises nucleic acid according to the invention in the form of nanoparticles. Nanoparticulate formulations can be obtained by various protocols and with various complexing compounds. Lipids, polymers, oligomers, or amphiphiles are typical constituents of nanoparticulate formulations.

As used herein, the term “nanoparticle” refers to any particle having a diameter making the particle suitable for systemic, in particular parenteral, administration, of, in particular, nucleic acids, typically a diameter of 1000 nanometers (nm) or less. In one embodiment, the nanoparticles have an average diameter in the range of from about 50 nm to about 1000 nm, preferably from about 50 nm to about 400 nm, preferably about 100 nm to about 300 nm such as about 150 nm to about 200 nm. In one embodiment, the nanoparticles have a diameter in the range of about 200 to about 700 nm, about 200 to about 600 nm, preferably about 250 to about 550 nm, in particular about 300 to about 500 nm or about 200 to about 400 nm.

In one embodiment, the polydispersity index (PI) of the nanoparticles described herein, as measured by dynamic light scattering, is 0.5 or less, preferably 0.4 or less or even more preferably 0.3 or less. The “polydispersity index” (PI) is a measurement of homogeneous or heterogeneous size distribution of the individual particles (such as liposomes) in a particle mixture and indicates the breadth of the particle distribution in a mixture. The PI can be determined, for example, as described in WO 2013/143555 A1.

As used herein, the term “nanoparticulate formulation” or similar terms refer to any particulate formulation that contains at least one nanoparticle. In some embodiments, a nanoparticulate composition is a uniform collection of nanoparticles. In some embodiments, a nanoparticulate composition is a lipid-containing pharmaceutical formulation, such as a liposome formulation or an emulsion.

In one embodiment, the pharmaceutical composition of the present invention comprises at least one lipid. Preferably, at least one lipid is a cationic lipid. Said lipid-containing pharmaceutical composition comprises nucleic acid according to the present invention. In one embodiment, the pharmaceutical composition according to the invention comprises RNA encapsulated in a vesicle, e.g., in a liposome. In one embodiment, the pharmaceutical composition according to the invention comprises RNA in the form of an emulsion. In one embodiment, the pharmaceutical composition according to the invention comprises RNA in a complex with a cationic compound, thereby forming, e.g., so-called lipoplexes or polyplexes. Encapsulation of RNA within vesicles such as liposomes is distinct from, for instance, lipid/RNA complexes. Lipid/RNA complexes are obtainable, e.g., when RNA is, e.g., mixed with pre-formed liposomes.

In one embodiment, the pharmaceutical composition according to the invention comprises RNA encapsulated in a vesicle. Such formulation is a particular particulate formulation according to the invention. A vesicle is a lipid bilayer rolled up into a spherical shell, enclosing a small space and separating that space from the space outside the vesicle. Typically, the space inside the vesicle is an aqueous space, i.e., comprises water. Typically, the space outside the vesicle is an aqueous space, i.e., comprises water. The lipid bilayer is formed by one or more lipids (vesicle-forming lipids). The membrane enclosing the vesicle is a lamellar phase, similar to that of the plasma membrane. The vesicle according to the present invention may be a multilamellar vesicle, a unilamellar vesicle, or a mixture thereof. When encapsulated in a vesicle, the RNA is typically separated from any external medium. Thus it is present in protected form, functionally equivalent to the protected form in a natural alphavirus. Suitable vesicles are particles, particularly nanoparticles, as described herein.

For example, RNA may be encapsulated in a liposome. In that embodiment, the pharmaceutical composition is or comprises a liposome formulation. Encapsulation within a liposome will typically protect RNA from RNase digestion. It is possible that the liposomes include some external RNA (e.g., on their surface), but at least half of the RNA (and ideally all of it) is encapsulated within the core of the liposome.

Liposomes are microscopic lipidic vesicles often having one or more bilayers of a vesicle-forming lipid, such as a phospholipid, and are capable of encapsulating a drug, e.g., RNA. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. Such phases may be present in nanoparticulate RNA formulations of the present invention.

Liposomes may be formed using standard methods known to the skilled person. Respective methods include the reverse evaporation method, the ethanol injection method, the dehydration-rehydration method, sonication or other suitable methods. Following liposome formation, the liposomes can be sized to obtain a population of liposomes having a substantially homogeneous size range.

In a preferred embodiment of the present invention, the RNA is present in a liposome which includes at least one cationic lipid. Respective liposomes can be formed from a single lipid or from a mixture of lipids, provided that at least one cationic lipid is used. Preferred cationic lipids have a nitrogen atom which is capable of being protonated; preferably, such cationic lipids are lipids with a tertiary amine group. A particularly suitable lipid with a tertiary amine group is 1,2-dilinoleyloxy-N,N-dimethyl-3-aminopropane (DLinDMA). In one embodiment, the RNA according to the present invention is present in a liposome formulation as described in WO 2012/006378 A1: a liposome having a lipid bilayer encapsulating an aqueous core including RNA, wherein the lipid bilayer comprises a lipid with a pKa in the range of 5.0 to 7.6, which preferably has a tertiary amine group. Preferred cationic lipids with a tertiary amine group include DLinDMA (pKa 5.8) and are generally described in WO 2012/031046 A2.

According to WO 2012/031046 A2, liposomes comprising a respective compound are particularly suitable for encapsulation of RNA and thus liposomal delivery of RNA. In one embodiment, the RNA according to the present invention is present in a liposome formulation, wherein the liposome includes at least one cationic lipid whose head group includes at least one nitrogen atom (N) which is capable of being protonated, wherein the liposome and the RNA have a N:P ratio of between 1:1 and 20:1. According to the present invention, “N:P ratio” refers to the molar ratio of nitrogen atoms (N) in the cationic lipid to phosphate atoms (P) in the RNA comprised in a lipid containing particle (e.g., liposome), as described in WO 2013/006825 A1. The N:P ratio of between 1:1 and 20:1 is implicated in the net charge of the liposome and in efficiency of delivery of RNA to a vertebrate cell.

In one embodiment, the RNA according to the present invention is present in a liposome formulation that comprises at least one lipid which includes a polyethylene glycol (PEG) moiety, wherein RNA is encapsulated within a PEGylated liposome such that the PEG moiety is present on the liposome's exterior, as described in WO 2012/031043 A1 and WO 2013/033563 A1.

In one embodiment, the RNA according to the present invention is present in a liposome formulation, wherein the liposome has a diameter in the range of 60-180 nm, as described in WO 2012/030901 A1.

In one embodiment, the RNA according to the present invention is present in a liposome formulation, wherein the RNA-containing liposomes have a net charge close to zero or negative, as disclosed in WO 2013/143555 A1.

In other embodiments, the RNA according to the present invention is present in the form of an emulsion. Emulsions have been previously described to be used for delivery of nucleic acid molecules, such as RNA molecules, to cells. Preferred herein are oil-in-water emulsions. The respective emulsion particles comprise an oil core and a cationic lipid. More preferred are cationic oil-in-water emulsions in which the RNA according to the present invention is complexed to the emulsion particles. The emulsion particles comprise an oil core and a cationic lipid. The cationic lipid can interact with the negatively charged RNA, thereby anchoring the RNA to the emulsion particles. In an oil-in-water emulsion, emulsion particles are dispersed in an aqueous continuous phase. For example, the average diameter of the emulsion particles may typically be from about 80 nm to 180 nm. In one embodiment, the pharmaceutical composition of the present invention is a cationic oil-in-water emulsion, wherein the emulsion particles comprise an oil core and a cationic lipid, as described in WO 2012/006380 A2. The RNA according to the present invention may be present in the form of an emulsion comprising a cationic lipid wherein the N:P ratio of the emulsion is at least 4:1, as described in WO 2013/006834 A1. The RNA according to the present invention may be present in the form of a cationic lipid emulsion, as described in WO 2013/006837 A1. In particular, the composition may comprise RNA complexed with a particle of a cationic oil-in-water emulsion, wherein the ratio of oil/lipid is at least about 8:1 (mole:mole).

In other embodiments, the pharmaceutical composition according to the invention comprises RNA in the format of a lipoplex. The term, “lipoplex” or “RNA lipoplex” refers to a complex of lipids and nucleic acids such as RNA. Lipoplexes can be formed of cationic (positively charged) liposomes and the anionic (negatively charged) nucleic acid. The cationic liposomes can also include a neutral “helper” lipid. In the simplest case, the lipoplexes form spontaneously by mixing the nucleic acid with the liposomes with a certain mixing protocol, however various other protocols may be applied. It is understood that electrostatic interactions between positively charged liposomes and negatively charged nucleic acid are the driving force for the lipoplex formation (WO 2013/143555 A1). In one embodiment of the present invention, the net charge of the RNA lipoplex particles is close to zero or negative. It is known that electro-neutral or negatively charged lipoplexes of RNA and liposomes lead to substantial RNA expression in spleen dendritic cells (DCs) after systemic administration and are not associated with the elevated toxicity that has been reported for positively charged liposomes and lipoplexes (see WO 2013/143555 A1). Therefore, in one embodiment of the present invention, the pharmaceutical composition according to the invention comprises RNA in the format of nanoparticles, preferably lipoplex nanoparticles, in which (i) the number of positive charges in the nanoparticles does not exceed the number of negative charges in the nanoparticles and/or (ii) the nanoparticles have a neutral or net negative charge and/or (iii) the charge ratio of positive charges to negative charges in the nanoparticles is 1.4:1 or less and/or (iv) the zeta potential of the nanoparticles is 0 or less. As described in WO 2013/143555 A1, zeta potential is a scientific term for electrokinetic potential in colloidal systems. In the present invention, (a) the zeta potential and (b) the charge ratio of the cationic lipid to the RNA in the nanoparticles can both be calculated as disclosed in WO 2013/143555 A1. In summary, pharmaceutical compositions which are nanoparticulate lipoplex formulations with a defined particle size, wherein the net charge of the particles is close to zero or negative, as disclosed in WO 2013/143555 A1, are preferred pharmaceutical compositions in the context of the present invention.

In a specific embodiment, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In a specific embodiment, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In a specific embodiment, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In a specific embodiment, the nanoparticles are lipoplexes comprising DOTMA and DOPE in a molar ratio of 2:1 to 1:2, preferably 2:1 to 1:1, and wherein the charge ratio of positive charges in DOTMA to negative charges in the RNA is 1.4:1 or less.

In a specific embodiment, the nanoparticles are lipoplexes comprising DOTMA and cholesterol in a molar ratio of 2:1 to 1:2, preferably 2:1 to 1:1, and wherein the charge ratio of positive charges in DOTMA to negative charges in the RNA is 1.4:1 or less.

In a specific embodiment, the nanoparticles are lipoplexes comprising DOTAP and DOPE in a molar ratio of 2:1 to 1:2, preferably 2:1 to 1:1, and wherein the charge ratio of positive charges in DOTAP to negative charges in the RNA is 1.4:1 or less.

In a specific embodiment, the RNA according to the invention is formulated in F12 or F5 liposomes, preferably F12 liposomes, wherein the term “F12” designates liposomes comprising DOTMA and DOPE in a molar ratio of 2:1 and lipoplexes with RNA which are formed using such liposomes and wherein the term “F5” designates liposomes comprising DOTMA and cholesterol in a molar ratio of 1:1 and lipoplexes with RNA which are formed using such liposomes.

In view of the ability of the RNA to be administered locally to an organ or tissue via an afferent blood vessel of the organ or tissue, and in view of the ability of the RNA to encode an agent, e.g., a peptide or polypeptide or siRNA, such that the agent is expressed and provides a therapeutic activity/effect, the methods of the invention are useful in methods of treating and/or preventing a disease or disorder, wherein the therapeutic effect/activity provided by the agent is useful in treating and/or preventing the disease or disorder. Exemplary diseases or disorders include, but are not limited to cancer, infectious disease, diseases of the nervous system, e.g., Alzheimer's disease, diseases of the kidney, diseases of the liver, diseases of the cardiovascular system, and diseases of the digestive system.

A “pharmaceutically active agent” has a positive or advantageous effect on the condition or disease state of a subject when administered to the subject in a therapeutically effective amount. Preferably, a pharmaceutically active agent, e.g., a peptide or polypeptide, has curative or palliative properties and may be administered to ameliorate, relieve, alleviate, reverse, delay onset of or lessen the severity of one or more symptoms of a disease or disorder. A pharmaceutically active agent may have prophylactic properties and may be used to delay the onset of a disease or to lessen the severity of such disease or pathological condition. The term “pharmaceutically active peptide or polypeptide” includes entire peptides or polypeptides, and can also refer to pharmaceutically active fragments thereof. It can also include pharmaceutically active analogs of a peptide or polypeptide.

The compounds provided herein, e.g., the RNA molecules encoding a pharmaceutically active agent, and compositions comprising the compounds provided herein, may be used alone or in combination with conventional therapeutic regimens.

The term “disease” or “disorder”, which can be used interchangeably, refers to an abnormal condition that affects the body of an individual. A disease is often construed as a medical condition associated with specific symptoms and signs. A disease may be caused by factors originally from an external source, such as infectious disease, or it may be caused by internal dysfunctions. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The terms “individual” and “subject” are used herein interchangeably. They refer to human beings, non-human primates or other mammals (e.g., mouse, rat, rabbit, dog, cat, cattle, swine, sheep, horse or primate) that can be afflicted with or are susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In preferred embodiments of the present invention, the “individual” or “subject” is a “patient”. The term “patient” means according to the invention a subject for treatment, in particular a diseased subject.

The term “infectious disease” refers to any disease which can be transmitted from individual to individual or from organism to organism, and is caused by a microbial agent (e.g., common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g., chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, influenza, animal diseases like foot-and-mouth disease, Peste de petits ruminants, porcine reproductive and respiratory syndrome virus or parasite diseases such as Chagas, Malaria and others.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the invention also comprises cancer metastases.

A disease to be treated according to the invention is preferably a disease involving cancer. Other diseases preferably include those that can be treated by local administration to an organ or tissue by administering to an afferent blood vessel of the organ or tissue an RNA encoding an agent having therapeutic activity, but which agent is considered to be too toxic to the body as a whole to be administered systemically.

The term “therapeutic treatment” or simply “treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The term “prophylactic treatment” or “preventive treatment” relates to any treatment that is intended to prevent a disease from occurring in an individual. The terms “prophylactic treatment” or “preventive treatment” are used herein interchangeably.

The teal's “protect”, “prevent”, “prophylactic”, “preventive”, or “protective” relate to the prevention and/or treatment of the occurrence and/or the propagation of a disease, e.g., tumor, in an individual. For example, a prophylactic administration of a cytotoxic agent, e.g., by administering a composition of the present invention, can protect the receiving individual from the development of a tumor. For example, by administering a composition of the present invention, the development of a disease can be stopped, e.g., leading to inhibition of the progress/growth of a tumor. This comprises the deceleration of the progress/growth of the tumor, in particular a disruption of the progression of the tumor, which preferably leads to elimination of the tumor.

By “being at risk” is meant a subject, i.e., a patient, that is identified as having a higher than normal chance of developing a disease compared to the general population. In addition, a subject who has had, or who currently has, a disease is a subject who has an increased risk for developing a disease, as such a subject may continue to develop a disease.

The term “in vivo” relates to the situation in a subject.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severity of the disease, the individual parameters of the patient, including age, physiological condition, size and weight, the duration of treatment, the type of an accompanying therapy (if present), and similar factors. Accordingly, the doses administered of the compositions described herein may depend on various of such parameters. In the case that a reaction in a patient is insufficient with an initial dose, higher doses may be used.

### Examples

A patient suffering from cancer, having a localized tumor in the brain, is administered an mRNA encoding a polypeptide agent, e.g., a cytokine or an antibody, through a catheter to the target organ, in this case the brain. The catheter is placed into the superficial femoral artery and is guided using x-rays after injection of an x-ray contrast agent. The catheter is navigated (guided) from the point of entry in the femoral artery into the descending aorta, into the great vessels and ultimately into a desired position within a vessel in the neck. The blood flow then carries the catheter into an afferent blood vessel of the brain. Once in place, the mRNA encoding the polypeptide agent, at a dose chosen to, e.g., not to exceed any threshold values in the serum associated with systemic toxic effects at any time, is administered/released from the catheter into the blood stream. Serum levels of the polypeptide agent in the patient can be determined by routine methods, e.g., by taking blood samples and performing ELISA.

We claim:

1. A method for locally expressing a peptide or polypeptide in an organ or tissue in a subject, comprising administering to an afferent blood vessel of the organ or tissue an RNA, which RNA encodes a peptide or polypeptide.

2. The method according to claim 1, wherein the afferent blood vessel feeds blood directly into the organ or tissue without feeding blood into other organs or tissues.

3. The method according to claim 1 or 2, wherein the afferent blood vessel is proximal/immediately upstream/close to the vascular bed of the organ or tissue.

4. The method according to any one of claims 1 to 3, wherein the afferent blood vessel is a part of the vascular bed of the organ or tissue.

5. The method according to any one of claims 1 to 4, wherein the peptide or polypeptide is one that is rapidly degraded in the blood stream.

6. The method according to any one of claims 1 to 5, wherein the peptide or polypeptide is one that is toxic.

7. The method according to any one of claims 1 to 6, wherein the peptide or polypeptide is one that is toxic, for example, unacceptably toxic, when administered systemically to the subject.

8. The method according to any one of claims 1 to 7, wherein the peptide or polypeptide is one that has been modified to have a shorter half-life in the blood stream.

9. The method according to any one of claims 1 to 8, wherein the peptide or polypeptide is one that has been modified to contain one or more additional protease cleavage sites.

10. The method according to any one of claims 1 to 9, wherein the peptide or polypeptide in one that has been modified to reduce the permeability of the peptide or polypeptide.

11. The method according to any one of claims 1 to 10, wherein the peptide or polypeptide is one that provides for a therapeutic effect.

12. The method according to any one of claims 1 to 10, wherein the peptide or polypeptide is one that can serve as a detectable moiety.

13. The method according to any one of claims 1 to 12, wherein the peptide or polypeptide is detectable in the organ or tissue but is not substantially detectable in other organs or tissues or in the blood stream following administration of the RNA.

14. The method according to any one of claims 1 to 13, wherein the tissue is tumor tissue.

15. The method according to any one of claims 1 to 13, wherein the organ is liver, thyroid, pancreas, kidney, lung, bladder, colon, ovary, testicle, prostate, breast, uterus, heart, stomach, or brain.

16. The method according to any one of claims 1 to 15, wherein the RNA is administered in combination with a vasoactive agent.

17. The method according to claim 16, wherein the vasoactive agent is one that enhances transcapillary vesicular transport across the capillary endothelial cell wall.

18. The method according to claim 16 or 17, wherein the vasoactive agent is administered before, after or together with the RNA, preferably within 5 minutes before or after administration of the RNA.

19. The method according to any one of claims 16 to 18, wherein the vasoactive agent is histamine or a vascular endothelial growth factor.

20. The method according to any one of claims 1 to 19, wherein the RNA is administered in a composition comprising the RNA and a pharmaceutically acceptable carrier.

21. The method according to any one of claims 1 to 20, wherein the subject is a mammal, preferably a human.

22. A method for treating, preventing or diagnosing a disease in an organ or tissue in a subject, comprising administering to an afferent blood vessel of the organ or tissue an RNA, which RNA encodes a peptide or polypeptide that is effective in treating, preventing or diagnosing the disease.

23. The method according to claim 22, wherein the disease is cancer or a tumor.

24. The method according to claim 23, wherein the tumor is a primary tumor or is a metastasis of a primary tumor.

25. The method according to any one of claims 22 to 24, wherein the peptide or polypeptide is one that is toxic.

26. The method according to any one of claims 1 to 25, wherein the RNA is comprised in a complex or vesicle.

27. The method according to claim 26, wherein the vesicle is a multilamellar vesicle, a unilammelar vesicle, or a mixture thereof.

28. The method according to claim 26 or 27, wherein the vesicle is a liposome.

29. The method according to claim 26, wherein the complex is a polyplex particle.

30. The method according to any one of claims 26 to 29, wherein the complex or vesicle further comprises a ligand for site-specific binding.

31. The method according to any one of claims 1 to 30, wherein the RNA is taken up by the cells of the organ or tissue and the peptide or polypeptide is expressed in the cells.

32. The method according to claim 31, wherein the peptide or polypeptide is secreted by the cells.

33. The method according to claim 32, wherein the peptide or polypeptide is distributed substantially only in the organ or tissue.

34. The method according to any one of claims 31 to 33, wherein the cells of the organ or tissue are endothelial cells.

35. A pharmaceutical composition comprising RNA encoding a peptide or polypeptide which is formulated for administration into an afferent blood vessel.
